SEQUENCE LISTING (12 sequences)

SEQ ID NO: 1 - DNA, 3236 bp, mouse
cactgaagctggtccacgtctataaacaggtgacactggctgcagcaaaaagccattcgatccacacaaattgatcttctatcatcttggaatctgaattgcagggaggagcagtatgtaagacgaccgtttaattcaggcattccgaaggcatgagcgcatggattctgtcaccaagcgtataaaaggaccctggcattgggaaacctatgacggactgtttttgctgtagaagtagggattttacagaagtctccttgaatttgccctgcctggggcagttttgcagaggaacctgccagagatttattggctggtcagtctcttgtgaaatagtatcatgtgagaaacagtttgtagaaaaaaactatacctgggaagacctttgcaacattgttccttccatgggccaagactcagttaggaggcataaatctgcccggaataaactaggccaggatacagccatgtttagttaataatttggttttagaattcacacaggcaggattggtttttttgtgtcttggcaagtggagcatatttaacatacaggcatgggaatcctgcctcttagcttttcccaccctcttgtctcaccaagttttttctctccaaaggtttccaggaatttctcattaatggctgatgcaaacttagtgaataataatgaatataaacaatgctcacctcaccaaaattatattatttgcagtcatttgtgataacacaaattttatcgcaatggttattatttaatttgtggccacacactgtggttatcttttgttgtggttgtttctgagaaaatgttcttggatatgtaagtgccaataccagtgtgaagtattgatcccgggcagcaaaatacagcctaaggtttgtaaacatcaattctatctcagttcatcagagggcctgagaagctgcggggcagtgtaaagtaaagtatgctgggctggtggtggtcagcctcccgcctgaagagtgaccagtgctggcccgacggatcgctgagatattctcccataatggcaaaaaaataggcagtttgatgtgacctgtttagtgtggctctcctcttttgagcatgtgttagcatttttattttatactcatccagtgaactctgctcttccaagtgtgttcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtgcgcagtaggattcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggagtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcaattactgtacaaaaacccagtgcagctgatgatgcaaagcagtctctctctgtgtacagtgccccacctatttaaaaatcacgtacaascccagaacactgtgaaacacttaacataagaacaaacgcagcgtctggattctttccaaggagagcagctttctccacaggaacacagtaacaaaagaggtccgccgccatccacacccagccaagacacctcagaggccatagggacaacctccttgctggccaacacctgctggagcaggggcacaggtcccagcaactgatcctcagtggatgggtccgcagtcaaagccttaatgggctctcttttgaaggggaaagaaagaatttcaagcttatgatatccaacattattatagttgatgagttagtaaattccaaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaatgaaaaaaaaaaaaaaaaaaaaaaaaaaaaaaaa

SEQ ID NO: 2 - DNA, 5752 bp, mouse
aagtgtaaataaaataaacatctaataaaaaaaattacataccatagaggaacaagataatttctgcccaacttcataccctccagcgtatagtgttgaggtttggtctgttgctgtgtattgtaatgtaatgttaaattctctacctgaaggtctaggcctacaagtgaattctcatgtttatagagttttgttgtgcaaaccttgttccttaatttaaaactatggttaaaaaacaaaacaaaactggctacagccaataactgaagggggttaccttgttgaaggggtggaaaagagagaggaggaagaagggagttcaagagaaggagaagaacaagaggagaggaggaagctgccacgaggggagatgggccatgagaacttggccaggagaaatagccagtatctggagtacaccactgaggaggtagccaggctagcagttagaagagtagattaggggttatttttcccccactccacatagttatcaaagccaaataaaataaccatagtctgagtctcatctatttgtaagctagttgggtataagattaatttggctgtactacagtttagatttctaacataggaactatcaaaaacttgctcaaacaagaacatgctgacaatattttaaaatgattatttatattgtttgcactttctaaagtttcttctaaatgttccatggtcaaattaaaaaatatacatattggctattaaattcgtctaagtggggctggagagatagctcagaggttaagagcactgactgctcttccagaggtcctgagttcaattcccagcgaccacatggtggctcacagccatctgtaatagataggatctgacgccctcttctggagtgtctgaagacagctacaatgtactcatatatattaaataaataatattagaaaattcttctaagtgtatcatttatagaatatttaatatataaagtaaatgcctcaggaaatataaacttggaattaaatcaaagaacttcatgagtagtgggccacaaaaaatgtgtaccaggggaagaccggagggaggggagaaggaagggatggagatagaattttgcctctgcattccttgggctggcacaggtataatgctgtgggaattgggaaactacaaggaagctgcaaagctgggcggaactcgtttccgcaagctgggctcatctaagtgtccatgcatggctgccacactgcagtgaactttaaaacatttgtgttccagagatgtagagatgctcacaatagtacaaaggcgggagggaggtatttccagactaagaggaagaaaaaccattgctgattaaacatctgcatatgagcgcccccacctccatacacacacacacacacacacacacacacacacaaccaaacagaacaaatacacatgcatgtctacagcctgcaggaacaaaatggtatgtctgtgaggaaccaggagatgcacaggtcctaacctctgtctcctacaagccctgaagtctggtcagggtcaaatgtacaaaagcaggctaaggaagctgtttagtgaaagatttttttcttcaactctaggaacaacctatttcctaggatttggagagtgctcaggaggaaacattcagacaactgatgctctctgtgtaccccagattcaggtattggggtagttagttgtgctcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtgcgcagcaggattcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggggtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacttgcccagaacactgtgaaacacttaacataagaacaaacgcagcgtctggattctttccaaggagagcagctttctccacaggaacacagtaacaaaagaggtccgccgccatccacacccagccaagacacctcagaggccatagggacaacctccttgctggccaacacctgctggagcaggggcacaggtcccagcaactgatcctcagtggatgggtctgcagccaaagccttaatgggctctcttttgaaggggaaagaaagaatttcaagcttatgatatccaatattattatagttgatgagttagtaaattccaaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaataaaaaaaaaaaaaaaaaaaaaaaaaaaaaaaaaattttttttttttttttttattccagagattaaagacactagatctttaaccttgaagggcaggcaagaggtcggcaatgctgtcaacatagaagtcagggaccattttcttcttgaacatgcagtcactttcctgattgctcttcacatcctcaaggctccggaattccgggggtgtggtgggctttgatctcaggactctggaggcagaagcaggcagatctctgtgaatatgaggccagcctgcactacacagagctccagaccagtcatggctacatcatgaaaccctgtctcaaaaagaaaataaaaactgttgtgtttctaccatagtgttaaactcagagtctgagtaatgtcgggctgacatgctcgggtgtttaacataccttcagctttgacgaggcgctgaacagtcaaagtctggccttggggagcggtggctgtgtttgtgctcaagtccaccgtgaaatcctgattgtgaatttggacaaccgtgtccttcttcttggccttccatgcaacctccaacttcatgttggtcattttgtcaaaacactgtgtgatgtttttatcaatatactgccattccacatatgtagagatgtagtctgcctggctttccttttctttagccaatcgaatgctcttgatcatgccctcaatctcatctctagcttttatcacgtctctgctaattcctgaaacttgaatcgaagttttcttctggttcatctcaatggtgatgttcagttccttctgaatctcattcagtttctcgtactcctccatgtcaaagtcactgacacactcatcgtcattggtgtaggaaagctgctctttggtaatcagttcctttagccaggagattgttttgttcacactgtctacccctgaaccacatacctggaaaactgtgtgctctattttcttttccaaaaccagggtgttctttttgggggaagcttgcttgggaaagccaagaaaggctaaagagaaaatggaaattaatgtttcttttactcccttcaacatcaaggttaggaatatgtatttcataaaagctaacaactcacaggcaatcttagacatcactgactgcttggcaggcgactgcttggggggagctggagagccttctctttctttcatgttgtcgtaaaaaaattgcagaatatggggctggaagataacaactttaactctcttcacagcctgcactgattttttctggacaaattcttcaatggcatctattatcgcttttgctactacgtttgggtcctgttgagcatttccttcaaaaacaaaaaaagcacatttttaaaaagtcaaggttaagatccacctgcaaaaaaaagctgcaatataagcgaggaattctagttgtcacaggaaataaaaatgtctgttcccactataatcaatgtagactgataatattatgccagcaaatagttttgaagtcctaggcacagtgggaggaggttttgttccacgctgttcataagccaataccccagcaaaagaccttaaaggacaacttgtaatttgggacattcacatctgtcctcttcatctgatctggctcccagtgtcactctctaacacggtccttagagggacaatttatccctgcctctgcttgatcttatgcatgtatctgtattcttccagccatccctggcgacctgatttttctaaggcacccaaaactgtaagctacttcttataatctataattctgagcatattagttagcctgagcctccaggatatctttcttccctatactcagtccagttttagctgcccagaaggattcaaagctgatctacgagtagatcactcctgtctacagcttgttccagatcttgtttctcaagccctggaagccatcagccaggtaagattgtaaaacaatccctttctaatcatgggtgtggcccaaagtgaatggccggaattc

SEQ ID NO: 3 - DNA, 475 bp, mouse
tgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacttgcccagaacactgtgaaacacttaacataagaacaaacgcagcgtctggattctttccaaggagagcagctttctccacaggaacacagtaacaaaagaggtccgccgccatccacacccagccaagacacctcagaggccatagggacaacctccttgctggccaacacctgctggagcaggggcacaggtcccagcaactgatcctcagtggatgggtctgcagccaaagccttaatgggctctcttttgaaggggaaagaaagaatttcaagcttatgatatccaatattattatagttgatgagttagtaaattccaaaaaaaaaa

SEQ ID NO: 4 - DNA, 20 bp, Artificial Sequence
  primer
agggctgtcaatcatgctgg

SEQ ID NO: 5 - DNA, 20 bp, Artificial Sequence
  primer
aaactcacggtcggtgcagc

SEQ ID NO: 6 - DNA, 24 bp, Artificial Sequence
  probe
attaaccctcactaaatgctgtat

SEQ ID NO: 7 - DNA, 30 bp, Artificial Sequence
  probe
cattatgctgagtgatatctttttttttcg

SEQ ID NO: 8 - DNA, 38 bp, Artificial Sequence
  probe
gaacatgtagcatatactccagacaacagatcatatgg

SEQ ID NO: 9 - DNA, 32 bp, Artificial Sequence
  probe
cagcttctccacaggaacacagtaacaaagag

SEQ ID NO: 10 - DNA, 35 bp, Artificial Sequence
  primer
ctatttcacaagagactgaccagccaataaatctc

SEQ ID NO: 11 - DNA, 7581 bp, unknown
  cDNA
  unsure (3159)...(3160): a or g or c or t/u, unknown, or other
  unsure (3383)...(3384): a or g or c or t/u, unknown, or other
  unsure 3392, 3397: a or g or c or t/u, unknown, or other
  unsure 3399,  3401: a or g or c or t/u, unknown, or other
  unsure 3404, 3406,  3408: a or g or c or t/u, unknown, or other
  unsure 3694: a or g or c or t/u, unknown, or other
  unsure 6791: a or g or c or t/u, unknown, or other
  unsure (7056)...(7057): a or g or c or t/u,  unknown or other
cgcccgggcaggtctgttggagggcagttggtcaacctgaccagagagagctgagctggaagaccccactgatggtgtgctgcctttcagtccaggaagaaagaaaggaaggattctgaggatttgggcaaagccacattcctggagaagtctgtatactgatgccaaacccaagagctgagctgctgatgaggcccagggagtagcccacgcgccctgagctgttggctagcaaggccttcctgctccatgtggcatggaaaaattatatggtttgacggatgaaaaggtgaaggcctatctttctctccatccccaggtattagatgaatttgtttctgaaagtgttagtgcagagactgtggaaaagtggctgaagaggaaaaccaacaaagcaaaagatgaaccatctcccaaggaagtcagcaggtaccaggatacgaatatgcagggagtcgtgtacgagctgaacagctacatagagcagcgcctggacacgggcggggacaaccacctgctcctctatgagctcagcagcatcatcaggatagccacaaaagccgacggatttgcactgtacttccttggagagtgcaataatagcctgtgtgtgttcataccacccgggatgaaggaaggccaaccccggctcatccctgcagggcccatcacccagggtaccaccatctctgcctacgtggccaagtctaggaagacgttgttggtagaggatatccttggggatgagcgatttcctcgaggtactggcctggaatcaggaacccgcatccagtctgttctttgcttgcccattgtcactgccattggagacttgattggcatccttgaactgtacaggcactggggcaaagaggccttctgcctcagccatcaggaggttgcaacagccaatcttgcttgggcttccgtagcaatacaccaggtgcaggtgtgtagaggtctcgccaaacagaccgaactgaatgacttcctactcgacgtatcaaagacatactttgataacatagttgccatagactctctacttgaacacatcatgatatatgcaaaaaatctagtgaacgccgaccgctgcgcgctcttccaggtggaccacaagaacaaggagctgtactcggacctgtttgacattggggaggagaaggaggggaagcccatcttcaagaagaccaaggagatcagattttccattgagaaagggattgctggtcaagtggcaagaacaggcgaagtcttgaacattcccgatgcctacgcggaccctcgctttaacagggaggtggacctgtacacaggctacaccacgaggaacattctgtgtatgcccatagtgagccgaggcagcgtgattggcgtggtgcagatggtgaacaagatcagcggtagcgccttctccaagacagacgagaacaacttcaagatgtttgctgtcttctgcgcactggccttgcactgtgctaacatgtaccacaggatccgccactcagaatgcatctacagggttaccatggagaagctttcctaccacagcatctgcacctccgaggagtggcaaggcctcatgcgcttcaacctaccagcacgcatctgccgggacatcgagctattccactttgacattggtcctttcgagaacatgtggcctgggatctttgtctacatgatccatcggtcttgtgggacatcctgttttgaacttgaaaaattgtgccgttttatcatgtctgtgaagaagaactatcggcgggttccttaccacaactggaagcatgcagtcacggtggcacactgcatgtatgccatacttcaaaacaacaatggcctcttcacagacctcgagcgcaaaggcctgctaattgcgtgtctgtgccatgacctggaccacaggggcttcagtaacagctacctgcagaagttcgaccaccccctggcggcgctgtactccacctccaccatggagcaacaccacttctcccagacggtgtccatccttcagctggaagggcacaatatcttctccaccctgagctccagcgagtacgagcaggtgctggagatcatccgcaaagccatcatcgccaccgacctcgccctatactttgggaacaggaagcagttggaggagatgtaccagacagggtcgctgaacctccacaaccagtcccatcgagaccgtgtcatcggcttgatgatgactgcctgtgatctttgctctgtgaccaaactatggccagttacaaaattgacagcgaatgatatatatgcagaattctgggctgagggtgatgagatgaagaagctgggcatacagcccattcctatgatggacagagacaagcgagatgaagtccctcaagggcagctcggattctacaatgctgtggccattccctgctataccaccttgacgcagatcctcccacccacagagcctctgctgaaggcctgcagggataacctcaatcagtgggagaaggtaattcgcggggaagagacagcaatgtggatttcaggcccaggcccggcgcctagcaagagcacacctgagaagctgaacgtgaaggttgaagactgatcctgaagtgacgtcctgatgtctgcccagcaaccgactcaacctgcttctgtgacttcgttctttttgttttcaaggggtgaaaaccccctgtcagaaggtaccgtcgcatatccatgtgaagcagacgactccctgcttgccgcacacacctcggacagtgagcaacccaggctctgccgtgttcagacgtcggctactccgtggctccacctgacctccgaatgctatttgctcccaggccagcactgcactgtctggagggggcagagaccacaggagaggttcttgcctgcatcctcccatgagggtgtggccagttccctagttctgtgccatgctgctgcttggtggcattggttaggaatgggacacacgccccttgttgtgaagtttacatgtgaccttcttataggttaactgagtttgtggcctggacacatgtaatgaaggtcacagtccacaggtgacagagaaatccaaactgttgattacaggtgcactacaggtatgctctttcagtctatctgggggcacataggtgagtctgctccactcagaannaagcatacctctgccctcatccaggggacacagggtacatcccaggcatcggggaactgaagctctcacttcaaaccatgtcaaagaattaaaacacctcccctccccctcactgtagccttcgacaactgcgccaatccctttatacaaagaaaataaaagtaaggcatataaatttcctccagcaagcaaatcttgtgggtaaaaaaaaagcatgtgaatnntaacaacntctanantntcncngnatgttatggcagaattttagtcacgtccaaaacaaaaagattattccagaagatacctcatcctatgcctgaaaggctccacagcatggcgtccgtctcccagggttctgatccgtctcctcacggtgcaatcaggcaggacagagaggagggctgcagggctaccacattgacccagaaggtatctcctctcaccattcagacatccataaggaatgccaaatgctgtattgaatagttctctgtgtgactttctagagaagccaggacaccctgagcctttccnggggaactctaaggagtcacaggttcacaccgtggggattttcaggatagcatggagacagagatccggtcgttgttctcactcgtgagccttgagaaggagagactgaccagaaacactcactcagcactctgcaggagcaggagaagatactttaagatgaatcttggatagattttgatacacccaataccatacacacaggagcttggcatttgcaaagtctattcagtttccttccgcgctctgacccacggttgtagcggagtgggctgaacactgtaacactgtacatgcgatttccccatgggcttctaaaatgtcaccatctcctcccctgctgtgtcctactccatttactggttacaaggtgatgtcaacaagagaagctatcacaacaccagggctgtgcacacgtgcacacacatgtatgcacaagcacacagatgtatgtacagcacacacacacacacacaccccaaaaggagagaaaaggaagaaaacatttataaaaagcgacagctaccccatatcaaaatagtctttcctgtaggaaacaggagctctccataaggaattatcatgagtgtgttctcccatcagtgcactctcccaggggtgctcactgaagctggtccacrtctataaacaggtgacactggctgcagcaaaaagccattcgatccacacaaattgatcttctatcatcttggaatctgaattgcagggaggagcagyatgtaagacgaccgtttaattcaggcattccgaaggcatgagcgcatggattctrtcaccaagcgtataaaaggaccctggcattgggaaacctatgacggactgtttttgctgtagaagtagggattttacagaagtctccttgratttgccctgcctggggcagttttgcagaggaacctgccagagatttattggctggtcagtctcttgtgaaatagtatcatgtgagaaacagtttgtagaaaaaaactatacctgggaagacctttgcaacattgttccttccatgggccaagactcagttaggaggcataaatctgcccggaataaactaggccaggatacagccatgtttagttaataatttggttttagaattcacacaggcaggattggtttttttgtgtcttggcaagtggagcatatttaacatacaggcatgggaatcctgcctcttagcttttcccaccctcttgtctcaccaagttttttctctccaaaggtttccaggaatttctcattaatggctgatgcaaacttagtgaataataatgaatataaacaatgctcacctcaccaaaattatattatttgcagtcatttgtgataacacaaattttatcgcaatggttattatttaatttgtggccacacactgtggttatcttttgttgtggttgtttctgagaaaatgttcttggatatgtaagtgccaataccagtgtgaagtattgatcccgggcagcaaaatacagcctaaggtttgtaaacatcaattctatctcagttcatcagagggcctgagaagctgcggggcagtgtaaagtaaagtatgctgggctggtggtggtcagcctccccttgccaagaagagagcaattgaatcctgtccccagctccctccacgcctgaagagtgaccagtgctggcccgacggatcgctgagatattctcccataatggcaaaaaaataggcagtttgatgtgacctgtttagtgtggctctcctcttttgagcatgtgttagcatttttattttatactcatccagtgaactctgctcttccaagtgtgttcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtggcagtaggatcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggagtgtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacaancccagaacactgtgaaacacttaacataagaaacaaacgcagcgtctggattctttccaaggagagcagctttctccacaggaacacagtaacaaaagaggtccgccgccatccacacccagccaagacacctcagaggccatagggacaacctccttgctggccaacacctgctggagcagggcacaggtcccagcaactgatcctcagtggatgggtccgcagtcaaagccttaatgggctctcttttgaaggggaaagaaanntttcaagcttatgatatccaacattattatagttgatgagttagtaaattccgaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaatgaaaaaaaaaaaaaac

SEQ ID NO: 12 - DNA, 7618 bp, mouse
  unsure 6810: a or g or c or t/u, unknown or other
  unsure (7075)...(7076): a or g or c or t/u, unknown or other
cgcccgggcaggtctgttggagggcagttggtcaacctgaccagagagagctgagctggaagaccccactgatggtgtgctgcctttcagtccaggaagaaagaaaggaaggattctgaggatttgggcaaagccacattcctggagaagtctgtatactgatgccaaacccaagagctgagctgctgatgaggcccagggagtagcccacgcgccctgagctgttggctagcaaggccttcctgctccatgtggcatggaaaaattatatggtttgacggatgaaaaggtgaaggcctatctttctctccatccccaggtattagatgaatttgtttctgaaagtgttagtgcagagactgtggaaaagtggctgaagaggaaaaccaacaaagcaaaagatgaaccatctcccaaggaagtcagcaggtaccaggatacgaatatgcagggagtcgtgtacgagctgaacagctacatagagcagcgcctggacacgggcggggacaaccacctgctcctctatgagctcagcagcatcatcaggatagccacaaaagccgacggatttgcactgtacttccttggagagtgcaataatagcctgtgtgtgttcataccacccgggatgaaggaaggccaaccccggctcatccctgcagggcccatcacccagggtaccaccatctctgcctacgtggccaagtctaggaagacgttgttggtagaggatatccttggggatgagcgatttcctcgaggtactggcctggaatcaggaacccgcatccagtctgttctttgcttgcccattgtcactgccattggagacttgattggcatccttgaactgtacaggcactggggcaaagaggccttctgcctcagccatcaggaggttgcaacagccaatcttgcttgggcttccgtagcaatacaccaggtgcaggtgtgtagaggtctcgccaaacagaccgaactgaatgacttcctactcgacgtatcaaagacatactttgataacatagttgccatagactctctacttgaacacatcatgatatatgcaaaaaatctagtgaacgccgaccgctgcgcgctcttccaggtggaccacaagaacaaggagctgtactcggacctgtttgacattggggaggagaaggaggggaagcccatcttcaagaagaccaaggagatcagattttccattgagaaagggattgctggtcaagtggcaagaacaggcgaagtcttgaacattcccgatgcctacgcggaccctcgctttaacagggaggtggacctgtacacaggctacaccacgaggaacattctgtgtatgcccatagtgagccgaggcagcgtgattggcgtggtgcagatggtgaacaagatcagcggtagcgccttctccaagacagacgagaacaacttcaagatgtttgctgtcttctgcgcactggccttgcactgtgctaacatgtaccacaggatccgccactcagaatgcatctacagggttaccatggagaagctttcctaccacagcatctgcacctccgaggagtggcaaggcctcatgcgcttcaacctaccagcacgcatctgccgggacatcgagctattccactttgacattggtcctttcgagaacatgtggcctgggatctttgtctacatgatccatcggtcttgtgggacatcctgttttgaacttgaaaaattgtgccgttttatcatgtctgtgaagaagaactatcggcgggttccttaccacaactggaagcatgcagtcacggtggcacactgcatgtatgccatacttcaaaacaacaatggcctcttcacagacctcgagcgcaaaggcctgctaattgcgtgtctgtgccatgacctggaccacaggggcttcagtaacagctacctgcagaagttcgaccaccccctggcggcgctgtactccacctccaccatggagcaacaccacttctcccagacggtgtccatccttcagctggaagggcacaatatcttctccaccctgagctccagcgagtacgagcaggtgctggagatcatccgcaaagccatcatcgccaccgacctcgccctatactttgggaacaggaagcagttggaggagatgtaccagacagggtcgctgaacctccacaaccagtcccatcgagaccgtgtcatcggcttgatgatgactgcctgtgatctttgctctgtgaccaaactatggccagttacaaaattgacagcgaatgatatatatgcagaattctgggctgagggtgatgagatgaagaagctgggcatacagcccattcctatgatggacagagacaagcgagatgaagtccctcaagggcagctcggattctacaatgctgtggccattccctgctataccaccttgacgcagatcctcccacccacagagcctctgctgaaggcctgcagggataacctcaatcagtgggagaaggtaattcgcggggaagagacagcaatgtggatttcaggcccaggcccggcgcctagcaagagcacacctgagaagctgaacgtgaaggttgaagactgatcctgaagtgacgtcctgatgtctgcccagcaaccgactcaacctgcttctgtgacttcgttctttttgttttcaaggggtgaaaaccccctgtcagaaggtaccgtcgcatatccatgtgaagcagacgactccctgcttgccgcacacacctcggacagtgagcaacccaggctctgccgtgttcagacgtcggctactccgtggctccacctgacctccgaatgctatttgctcccaggccagcactgcactgtctggagggggcagagaccacaggagaggttcttgcctgcatcctcccatgagggtgtggccagttccctagttctgtgccatgctgctgcttggtggcattggttaggaatgggacacacgccccttgttgtgaagtttacatgtgaccttcttataggttaactgagtttgtggcctgggacacatgtaatgaaggtcacagtccacaggtgacagagaaatccaaactgttgattacaggtgcactacaggtatgctctttcagtctatctgggggcacataggtgagtctgctccactcagaaggaagcatacctctsccctcatccaggggacacagggtacatcccaggcatcggggaactgaagctctcacttcaaaccatgtcaaagaattaaaacacctcccctccccctcactgtagccttcggcaactgcgccaatccctttatacaaagaaaatataagtaaggcatataaatttcctccagcaagcaaatcttgtgggtaaaaaaaaaaaatgtgaattttaacaacctctatattttcactgtatgttatggcagaattttagtcacgtccaaaacaaaagattattccagaagatacctcatcctatgcctgaaagctccacagcatggcgtccgtctcccagggttctgatccgtctcctcacggtgcaatcaggcaggacaggaggaggtgcagggctaccacattgacccagatggtatctcctctcaccattcagacatccataaggaatgccaaatgctgtattgaatagttctcctgtgtgactttctagagaagccaggacacccctgagcctttcctgggaactcctaaggaagtcacaggttcacaccgtggggattttcaggatagcatggagaccagagaatcccggttcggttgttctcactcggtgagccttgagaaggaagagactgaccagaaacactcactcagcactctggcaggagcaggagaagatactttaagatgaatctttgggatagattttgatacacccaataccatacacacaggagcttggcatttgcaaagtctattcagtttccttccacactctgacccacggttgtagcggagtgggctgaacactgtaacactgtacatgcgatttccccatgggcttctaaaatgtcaccatctcctcccctgctgtgtcctactccatttactggttacaaggtgatgtcaacaagagaagctatcacaacaccagggctgtgcacacgtgcacacacatgtatgcacaagcacacagatgtatgtacagcacacacacacacacacaccccaaaaggagagaaaaggaagaaaacatttataaaaagcgacagctacccccatattcaaaaatagttcttttccctgtagggaaacaggtagctctccataaggaaattatcatgagtgtgttctcccatcagtgcacttctcccaggggtgctcactgaagctggtccacgtctataaacaggtgacactggctgcagcaaaaagccattcgatccacacaaattgatcttctatcatcttggaatctgaattgcagggaggagcagcatgtaagacgaccgtttaattcaggcattccgaaggcatgagcgcatggattctgtcaccaagcgtataaaaggaccctggcattgggaaacctatgacggactgtttttgctgtagaagtagggattttacagaagtctccttggatttgccctgcctggggcagttttgcagaggaacctgccagagatttattggctggtcagtctcttgtgaaatagtatcatgtgagaaacagtttgtagaaaaaaactatacctgggaagacctttgcaacattgttccttccatgggccaagactcagttaggaggcataaatctgcccggaataaactaggccaggatacagccatgtttagttaataatttggttttagaattcacacaggcaggattggtttttttgtgtcttggcaagtggagcatatttaacatacaggcatgggaatcctgcctcttagcttttcccaccctcttgtctcaccaagttttttctctccaaaggtttccaggaatttctcattaatggctgatgcaaacttagtgaataataatgaatataaacaatgctcacctcaccaaaattatattatttgcagtcatttgtgataacacaaattttatcgcaatggttattatttaatttgtggccacacactgtggttatcttttgttgtggttgtttctgagaaaatgttcttggatatgtaagtgccaataccagtgtgaagtattgatcccgggcagcaaaatacagcctaaggtttgtaaacatcaattctatctcagttcatcagagggcctgagaagctgcggggcagtgtaaagtaaagtatgctgggctggtggtggtcagcctccccttgccaagaagagagcaattgaatcctgtccccagctccctccacgcctgaagagtgaccagtgctggcccgacggatcgctgagatattctcccataatggcaaaaaaataggcagtttgatgtgacctgtttagtgtggctctcctcttttgagcatgtgttagcatttttattttatactcatccagtgaactctgctcttccaagtgtgttcatgtatgtgctagatatattagcacagcctgccttctgctgcacaacgccttagagacccggcctttcaatgagcttagcttgtgctctgtttctgctctcttaggtctaaactatggtgtcagttttaatagaacaaaagtatgcatcttgccttggcttgagccttttcgttttcaatgctgacttctcccctttctctcctgtgctcaccttacctttccagagtgtaagggacaacttttaaggaggcgtgtccctggtaggggcatccctgttcaccaggtgcctgtcatcaccccacttgactgacatctaccctggtgactatgggttcctcttgtttgtagggaacggtggctccaggtggaggcatcaatctgttgggttctggttcccggctgcctttggttttgaaagtctcttctctgtatattcctaccctgcatttgctttgtgtggtgctgatgctgtggcagtaggatcttggatgactctccatcagtcacagactccccctgttgcaaagtgtcaggctgactcgacagtcaccgtaaaatctgagtcagtcacacacaggctgtcagccacggcttccacttgcatggctattctattttcacacgtgagtttctgttgctggctggctgactggcattatctatgctaagttgaaatcaggagtgtgcccagcagagcccatcattctcactgtctttgaaacaaagctgtacggtttgatcgatgaacgtatttaaagcatttcatgcaatgacaaagtgctcagtagtggaaggcaggctgtgaccagtctgcctgctccttactataattgtgaggatttgttactggaacagtacatggaggcctgaccttgtgggggcacagggtggaaccttagctgaatatagtgtgtgtctcaagaggaagtcagggtactagctcagtgctcaatctccaggtactatatatacatttgcccgttttatctctaatgtgaaataaatccccaaacacttgtttatcgtgtagcgtacctaaaagactattctattatgggtgtccccactttcttggtttggtcaccccgatcccccggtcttctgctgtatctagaacagtgactataaatgatgtatgggaatagtgtttccatatgatctgttgtctggagtatatgctacatgttcatttactgtacaaaaacccagtgcagctgatgatgcaaagcagtctctctctgtgtacagtgccccacctatttaaaaatcacgtacaancccagaacactgtgaaacacttaacataagaaacaaacgcagcgtctggattctttccaaggagagcagctttctccacaggaacacagtaacaaaagaggtccgccgccatccacacccagccaagacacctcagaggccatagggacaacctccttgctggccaacacctgctggagcagggcacaggtcccagcaactgatcctcagtggatgggtccgcagtcaaagccttaatgggctctcttttgaaggggaaagaaanntttcaagcttatgatatccaacattattatagttgatgagttagtaaattccgaaaaaaaaagatgattttatatgtatgacataaaaaaaatctttgtaaagtgcgcaagtgcaataatttaaagaggtcttatctttgcatttataaattataaatattgtacatgtgtgtaatttttcatgtattcatttgcagtctttgtatttaaaaaaactttactgttatgtttgtataatagaacattaatcatttattataactcagacaaggtgtaaataaattcataattcaaacagccagtatatatgcatatatgggtgttacattgcaaaaatctctatctttgttctattcacatgcttaaagaagtaagaaatcttttgtggatatgtaattatacatataaagtatatatatatgtatgatacatgaaatatatttagaaatgttcataattttaatggatattctttggtgtgaataattgaatacaacatttttaaaatgaaaaaaaaaaaaaaaaaaaaaaaaaaaaaaaaa

(19) United States
(12) Patent Application Publication
(10) Pub. No.: US 2007/0060606 A1
(43) Pub. Date: Mar. 15, 2007

(54) Compounds and methods for modulating phosphodiesterase 10A

(76) Inventors: Harold A. Robertson, Halifax (CA); Eileen M. Denovan-Wright, Halifax (CA); Donald Weaver, Halifax (CA)

Correspondence Address: BROMBERG & SUNSTEIN LLP, 125 SUMMER STREET, BOSTON, MA, 02110-1618, US

(21) Appl. No.: 11/508,755

(22) Filed: Aug. 23, 2006

Related U.S. Application Data
(63) Continuation-in-part of application No. 10/659,770, filed on Sep. 10, 2003.
Application No. 10/659,770 is a continuation of application No. 09/680,208, filed on Oct. 6, 2000, now abandoned.
(60) Provisional application No. 60/158,043, filed on Oct. 7, 1999.
(60) Provisional application No. 60/217,765, filed on Jul. 12, 2000.

(51) Int. Cl. A61K 31/4741; C07D 491/02; A61K 31/428; C07D 417/02

(52) U.S. Cl. 514/291; 546/80; 514/367; 548/183

(57) ABSTRACT

Novel compounds that modulate PDE10A, a gene that is primarily expressed in medium spiny neurons of the mammalian striatum and has been found to inhibit striatal output by reducing spiny medium excitability and effect cAMP and cGMP signalling cascades in vivo in rats, and methods for treating psychosis and schizophrenia using the novel compounds. Methods for screening for further compounds to modulate PDE10A activity are also taught.

### CROSS REFERENCE TO RELATED APPLICATIONS

This patent is a continuation-in-part application of U.S. patent application Ser. No. 10/659,770 filed Sep. 10, 2003 (now U.S. Published Application No. 2004/0152106, published Aug. 5, 2004) which is a continuation from U.S. patent application Ser.No. 09/680,208 filed Oct. 6, 2000, which claims priority from U.S. provisional application No. 60/158,043 filed Oct. 7, 1999, and U.S. provisional application No. 60/217,765 filed Jul. 12, 2000, all of which are hereby incorporated by reference herein in their entirety.

### TECHNICAL FIELD AND BACKGROUND ART

The present invention relates to compounds and methods for modulating, PDE10A, a polynucleotide whose expression is linked to psychosis and schizophrenia, and which is down-regulated during the development of CAG repeat disorders. The unique location of PDE10A to brain regions having dopaminergic input suggests a potential role in neurological and psychiatric illness. The present invention describes methods for modulating phosphodiesterase 10A (PDE10A) with novel benzofuranylpyridone and indole compounds and their salts and derivatives.

### BACKGROUND OF THE INVENTION

Very few if any effective treatments exist for neurological disorders characterized by progressive cell loss, known as neurodegenerative diseases, as well as those involving acute cell loss, such as stroke and trauma. In addition, few effective treatments exist for neurological disorders such as psychosis, which has been linked to altered striatal function relating to changes in expression of the enzyme PDE10A (see J. A. Siuciak, et al. (2006) Genetic deletion of the striatum-enriched phosphodiesterase PDE10A: Evidence for altered striatal function. Neuropharmacology. 51, 374-385, incorporated by reference herein), schizophrenia or other CNS disorders. Striatal dysfunction is implicated in a number of CNS disorders including psychosis, schizophrenia, obsessive-compulsive disorders, Parkinson's disease and Huntington's disease. The recent Siuciak results with PDE10A knock-out mice (above) provide evidence that PDE10A functions to inhibit striatal output by reducing spiny medium excitability. PDE10A is selectively expressed in dopaminoreceptive medium spiny neurons, and considerable data suggests that cAMP and cGMP signalling pathways play significant roles in the regulation of medium spiny neuron excitability. Additional studies with papaverine, a potent inhibitor of PDE10A, confirm that PDE10A regulates both cAMP and cGMP in vivo in rats (see J. A. Siuciak, et al. (2006) Inhibition of the striatum-enriched phosphodiesterase PDE10A: A novel approach to the treatment of psychosis. Neuropharmacology. 51, 386-396, incorporated by reference herein).

Other studies with rats administered subchronic doses of phencyclidine (PCP), an N-methyl-D-aspartate (NMDA) receptor antagonist that mimics and exacerbates the symptoms of schizophrenia, showed that persistent suppression of NMDA with PCP produced enduring structural changes in neocortical and limbic regions of the brain, similar to what is reported in schizophrenia. If acute treatment with the PDE10A inhibitor papaverine occurred immediately prior to the subchronic administration of PCP, the schizophrenia-like symptoms and neocortical/limbic changes were attenuated. See J. S. Rodefer et al.(2005) PDE10A inhibition reverses subchronic PCP-induced deficits in attentional set-shifting in rats. See Eur J Neurosci. (2005) 21, 1070-1076, incorporated by reference herein.

The papaverine results with PDE10A showing that PDE10A regulates cAMP and cGMP in rats, coupled with the PDE10A knock-out mice results showing that altered PDE10A activity affects striatal function and the papaverine attenuation of schizophrenia-like symptoms in mice administered subchronic PCP all indicate that modulators of PDE10A activity should be effective therapeutic agents in the treatment of diseases associated with striatal regions of the brain.

Huntington's disease (HD) is an inherited neurological disorder that is transmitted in autosomal dominant fashion. HD results from genetically programmed degeneration of neurons in certain areas of the brain. Huntington's disease is caused by a mutation of the gene IT-15 that codes for the protein huntingtin. The huntingtin gene contains a polymorphic stretch of repeated CAG trinucleotides that encode a polyglutamine tract within huntingtin. If this tract exceeds 35 in number, Huntington's disease results. Huntington's disease is only one of a number of neurological diseases which are characterised by these polyglutamine repeats. Schizophrenia, dementia, including Lewy Body disease, and stroke, trauma, and Parkinson's disease also affect the basal ganglia.

Huntingtin has no sequence similarity to known proteins. The function of the normal or mutated HD form of huntingtin has not been defined by the prior art. It is evident, however, that the expression of the HD form of huntingtin leads to progressive and selective neuronal loss. It has been demonstrated that the GABA- and enkephalin-containing medium spiny projection neurons of the caudate-putamen eventually die as a result of HD. Patients with minimal cell loss, however, still present with motor and cognitive symptoms suggesting that neuronal dysfunction, and not simply cell loss, contribute to the symptoms of HD. The motor symptoms of HD include the development of chorea, dystonia, bradykinesia and tremors. Voluntary movements may also be affected such that there may be disturbances in speech and degradation of fine motor co-ordination. In addition to motor decline, emotional disturbances and cognitive loss are also evident during the progression of HD.

Despite the fact that huntingtin is ubiquitously expressed, HD specifically affects cells of the basal ganglia, structures deep within the brain that have a number of important functions, including co-ordinating movement. The basal ganglia includes the caudate nucleus, the putamen, the nucleus accumbens and the olfactory tubercule. HD also affects the brain's outer surface, or cortex, which controls thought, perception, and memory. The mechanism by which only a small group of neurons in the striatum and cortex are rendered vulnerable to this ubiquitously expressed mutant protein is not known. There are no effective treatments for Huntington's disease.

Huntington's disease is widely believed to be a gain-of function disorder but neither the normal function nor the gained function of huntingtin is known. Because the function for huntingtin is not known, there is little insight into the disease process. It was believed that huntingtin was related to neuronal intranuclear inclusions (NII). However, recent results have cast doubt on our understanding of the role of the NII in Huntington's disease or in other CAG repeat disorders.

The development of a mouse carrying the 5′ end of the human Huntington's disease gene (the promoter and first exon) was an important step in the development of the tools that will allow us to understand the finction (and gain-of-function) associated with huntingtin. R6/2 mice exhibit a rapidly progressing neurological phenotype with onset at about 8 weeks. This phenotype includes a movement disorder characterised by shuddering, resting tremor, epileptic seizures and stereotyped behaviour. These symptoms suggest that the function of the basal ganglia is affected by the expression of the human exon 1 transgene prior to neuronal cell death. By 12 weeks the affected mice have significantly reduced brain weights and they die by about 13 weeks of age. Neuronal intranuclear inclusions (NII) develop at about 4 weeks. As is observed in human Huntington's disease patient, the R6/2 mice show changes in neuronal receptors. The present inventors have also demonstrated that changes in the expression of DARPP-32 and cannabinoid receptors change over time in HD mice; such changes have also been observed in human Huntington's disease patients (unpublished results). The loss of the cannabinoid receptor is one of the earliest documented changes that occur prior to neuronal degeneration in human HD patients. The R6/2 model, therefore, mimics the early phases of HD, a point in disease development where intervention would be most appropriate.

Human PDE10 was recently identified by identification of cDNA fragments published on the National Center for Biotechnology Information (NCBI) Expressed Sequence Tags (EST) database (Loughney et al., WO99/42596). While PDE10 was found to share homology with known PDEs, no function could be identified for PDE10.

### SUMMARY OF THE INVENTION

One particular embodiment in accordance with the presently claimed invention provides a compound of formula I,

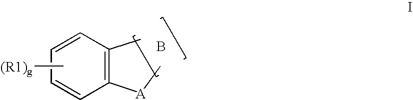

and pharmaceutically acceptable salts thereof, wherein
A is NR, O or S;
R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl;
g is 0, 1, 2, 3, or 4; and
B is a ring forming a fused ring system with the ring containing A and is selected from; 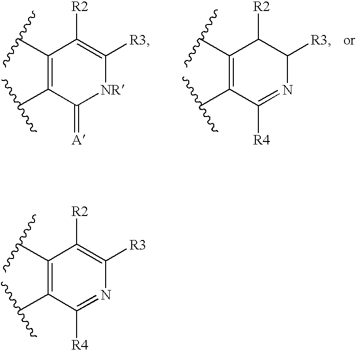
wherein A′ is as described above for A and NR′ is as described above for NR,

R1, R2, R3 and R4 are independently selected from:
(i) hydrogen, C₁ to C₅ alkyl, OH, NH₂, C₁ to C₅ alkylamino, di(C₁ to C₅ alkyl)amino, C₁ to C₅ alkylcarbonyl, C₁ to C₅ alkyloxycarbonyl, C₁ to C₅ alkylcarbonyloxy, carboxyl, C₁ to C₅ alkyl phosphonate, C₁ to C₅ alkenyl phosphonate, C₁ to C₅ alkyl phosphate, C₁ to C₅ alkenyl phosphate, C₁ to C₅ alkyl sulfonate, C₁ to C₅ alkenyl sulfonate, halo, halo(C₁ to C₅)alkyl, amino(C₁ to C₅)alkyl, hydroxyl(C₁ to C₅)alkyl, (C₁ to C₅)alkoxyl) C₁ to C₅ alkyl, NO₂, C₁ to C₅ alkylthio, SO₃H, PO₄, PO₃H, NH₄, C₂ to C₅ alkenyl, C₂ to C₅ alkenyloxy, C₂ to C₄ alkenylamino, di(C₂ to C₅ alkenylcarbonyl), C₂ to C5₄ alkenyloxycarbonyl, C₂ to C₄ alkylcarbonyloxy, halo(C₂ to C₅)alkynyl, amino(C₂ to C₅)alkenyl, hydroxy(C₂ to C₅)alkenyl, (C₁ to C₅ alkoxy) C₂ to C₅ alkenyl, C₂ to C₅ alkenylthio, C₂ to C₄ alkynyl, C₂ to C₅ alkynyloxy, C₂ to C₅ alkynylamino, di(C₂ to C₅ alkynyl)amino, C₂ to C₅ alkynylcarbonyl, C₂ to C₅ alkynyloxycarbonyl, C₂ to C₅ alkynylcarbonyloxy, halo(C₂ to C₅)alkynyl, amino(C₂ to C₅)alkynyl, hydroxy(C₂ to C₅)alkynyl, (C₁ to C₅ alkoxy) C₂ to C₅ alkynyl;
(ii) C₁ to C₅ alkoxy; and

(iii) aryl and arylalkyl.

Related embodiments provide a compound of formula I selected from the group consisting of 1,2-dihydro-1-oxobenzofuro[2,3-c]pyridine-7-carboxylic acid; 1,2-dihydro-1-oxobenzofuro[2,3-c]pyridine-6-carboxylic acid; (2E)-3-(1,2-dihydro-1-oxobenzofuro[2,3-c]pyridin-6-yl)acrylic acid; and 1,2-dihydro-1-oxobenzofuro[2,3-c]pyridine-8-carboxylic acid, and pharmaceutically acceptable salts thereof.

Still another particular embodiment in accordance with the presently claimed invention provide a compound of formula II

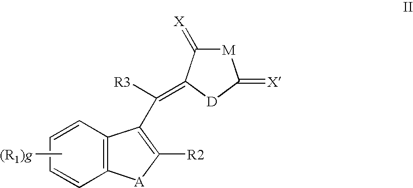

and pharmaceutically acceptable salts thereof, wherein

A, D and M are independently NR, 0 or S;

R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl;

g is 0, 1, 2, 3 or 4; and

X and X′ are independently O or S;

R1, R2 and R3 are independently selected from:
(i) hydrogen, C₁ to C5 alkyl, OH, NH₂, C₁ to C₅ alkylamino, di(C₁ to C₅ alkyl)amino, C₁ to C₅ alkylcarbonyl, C₁ to C₅ alkyloxycarbonyl, C₁ to C₅ alkylcarbonyloxy, carboxyl, C₁ to C₅ alkyl phosphonate, C₁ to C₅ alkenyl phosphonate, C₁ to C₅ alkyl phosphate, C₁ to C₅ alkenyl phosphate, C₁ to C₅ alkyl sulfonate, C₁ to C₅ alkenyl sulfonate, halo, halo(C₁ to C₅)alkyl, amino(C₁ to C₅)alkyl, hydroxyl(C₁ to C₅)alkyl, (C₁ to C₅)alkoxyl) C₁ to C₅ alkyl, NO₂, C₁ to C₅ alkylthio, SO₃H, PO₄, PO₃H, NH₄, C₂ to C₅ alkenyl, C₂ to C₅ alkenyloxy, C₂ to C₄ alkenylamino, di(C₂ to C₅ alkenylcarbonyl), C₂ to C5₄ alkenyloxycarbonyl, C₂ to C₄ alkylcarbonyloxy, halo(C₂ to C₅)alkynyl, amino(C₂ to C₅)alkenyl, hydroxy(C₂ to C₅)alkenyl, (C₁ to C₅ alkoxy) C₂ to C₅ alkenyl, C₂ to C₅ alkenylthio, C₂ to C₄ alkynyl, C₂ to C₅ alkynyloxy, C₂ to C₅ alkynylamino, di(C₂ to C₅ alkynyl)amino, C₂ to C₅ alkynylcarbonyl, C₂ to C₅ alkynyloxycarbonyl, C₂ to C₅ alkynylcarbonyloxy, halo(C₂ to C₅)alkynyl, amino(C₂ to C₅)alkynyl, hydroxy(C₂ to C₅)alkynyl, (C₁ to C₅ alkoxy) C₂ to C₅ alkynyl;

(ii) C₁ to C₅ alkoxy; and
(iii) aryl and arylalkyl.

Related embodiments provide a compound of formula II selected from the group consisting of (Z)-5-((1H-indol-3-yl)methylene)-2-thiooxazolidin-4-one; (Z)-5-((1H-indol-3yl)methylene)oxazolidine-2,4-dione; (Z)-5-((1H-indol-3-yl)methylene)thiazolidine-2,4-dione; (Z)-5-((1H-indol-3-yl)methylene)-2-thiothiazolidin-4-one, and pharmaceutically acceptable salts thereof.

One particular embodiment of the invention provides a modulator of PDE10A and dopamine binding to receptor molecules in neuronal tissue of the striatum, wherein the modulator comprises a compound of formula I or II.

In another particular embodiment of the present invention there is provided a method for treating a central nervous system (CNS) disorder associated with the striatal region of the brain, the method comprising administering an effective dose of a pharmaceutical formulation comprising a compound of formula I to a patient in need thereof exhibiting symptoms of a CNS disorder so as to attenuate said symptoms. Another embodiment provides a method for treating a central nervous system (CNS) disorder associated with the striatal region of the brain, the method comprising administering an effective dose of a pharmaceutical formulation comprising a compound of formula II to a patient in need thereof exhibiting symptoms of a CNS disorder so as to attenuate said symptoms. The symptoms may be clinical, including positive (i.e. hallucinations, delusions, racing thoughts), negative (i.e. apathy, lack of emotion, poor or nonexistant social functioning), and cognitive (disorganized thoughts, difficulty concentrating and/or following instructions, difficulty completing tasks, memory problems) symptoms. Symptoms are frequently characterized by profound disruption in cognition and emotion, affecting the most fundamental human attributes: language, thought, perception, affect, and sense of self. The array of symptoms, while wide ranging, may also include psychotic manifestations, such as hearing internal voices or experiencing other sensations not connected to an obvious source (hallucinations) and assigning unusual significance or meaning to normal events or holding fixed false personal beliefs (delusions). Psychotic symptoms may include delusions, hallucinations, disorganized speech, grossly disorganized or catatonic behavior or other psychotic behavior.

Related embodiments provide a method for treating a central nervous system (CNS) disorder associated with the striatal region of the brain by administering an effective dose of a pharmaceutical formulation comprising a compound of formula I or formula II, wherein the compound of formula 1 or formula II is selected from the group consisting of: 1,2-dihydro-1-oxobenzofuro[2,3-c]pyridine-7-carboxylic acid; 1,2-dihydro-1-oxobenzofuro[2,3-c]pyridine-6-carboxylic acid; (2E)-3-(1,2-dihydro-1-oxobenzofuro[2,3-c]pyridin-6-yl)acrylic acid; 1,2-dihydro-1-oxobenzofuro[2,3-c]pyridine-8-carboxylic acid; (Z)-5-((1H-indol-3-yl)methylene)-2-thiooxazolidin-4-one; (Z)-5-((1H-indol-3-yl)methylene)oxazolidine-2,4-dione; (Z)-5-((1H-indol-3-yl)methylene)thiazolidine-2,4-dione; (Z)-5-((1H-indol-3-yl)methylene)-2-thiothiazolidin-4-one, and pharmaceutically acceptable salts thereof.

Another embodiment provides a method for modulating PDE10A expression in a subject, the method comprising administering a compound of formula I in a pharmaceutical formulation, measuring isolated PDE1 OA mRNA from a sample of blood from the subject using a quantitative replicative procedure such as QPCR, and comparing the level of isolated mRNA from blood from the subject before and after administering the compound of formula I.

Another particular embodiment provides a method for modulating PDE10A expression in a subject, the method comprising administering a compound of formula II in a pharmaceutical formulation, measuring isolated PDE10A mRNA from a sample of blood from the subject using a quantitative replicative procedure such as QPCR, and comparing the level of isolated mRNA from blood from the subject before and after administering the compound of formula II. Related embodiments provide a method for modulating PDE10A expression in a subject comprising administering a compound of formula I or formula II, wherein the compound of formula I or formula II is selected from the group consisting of: 1, 2-dihydro-1-oxobenzofuro[2,3-c]pyridine-7-carboxylic acid; 1,2-dihydro-1-oxobenzofuro[2,3-c]pyridine-6-carboxylic acid; (2E)-3-(1,2-dihydro-1-oxobenzofuro[2,3-c]pyridin-6-yl)acrylic acid; 1,2-dihydro-1-oxobenzofuro[2,3-c]pyridine-8-carboxylic acid; (Z)-5-((1H-indol-3-yl)methylene)-2-thiooxazolidin-4-one; (Z)-5-((1H-indol-3-yl)methylene) oxazolidine-2,4-dione; (Z)-5-((1H-indol-3-yl)methylene)thiazolidine-2,4-dione; (Z)-5-((1H-indol-3-yl)methylene)-2-thiothiazolidin-4-one, and pharmaceutically acceptable salts thereof.

The present invention provides the function and uses of a nucleotide segment, PDE10A, and compounds which inhibit or promote the development of CAG repeat disorders such as Huntington's Disease.

The invention teaches a method for identifying a compound which inhibits or promotes a CAG repeat disorder, comprising the steps of: (a) selecting a control animal having PDE10A and a test animal having PDE10A; (b) treating said test animal using a compound; and (c) determining the relative quantity of RNA corresponding to PDE10A, as between said animals. In an embodiment, the animal is a mammal, preferably a mouse, and preferably a transgenic mouse. In an embodiment, the CAG repeat disorder is Huntington's disease.

The invention also teaches a method for identifying a compound which inhibits or promotes a CAG repeat disorder, comprising the steps of: (a) selecting a host cell containing PDE10A; (b) cloning said host cell and separating said clones into a test group and a control group; (c) treating said test group using a compound; and (c) determining the relative quantity of RNA corresponding to PDE10A, as between said test group and said control group. In an embodiment, the CAG repeat disorder is Huntington's disease.

The invention further teaches a method for detecting the presence of or the predisposition for a CAG repeat disorder, said method comprising determining the level of expression of RNA corresponding to PDE10A in an individual relative to a predetermined control level of expression, wherein a decreased expression of said RNA as compared to said control is indicative of a CAG repeat disorder. Preferably, the expression is measured by in situ hybridization, fluorescent in situ hybridization, polymerase chain reaction, or DNA fingerprinting technique. In an embodiment, the CAG repeat disorder is Huntington's disease.

The invention further teaches compositions for treating a CAG repeat disorder comprising a compound which modulates PDE10 expression and a pharmaceutically acceptable carrier. The compound can be selected from the group consisting of: quinpirole, alloxan, miconazole nitrate, MDL-12330A and tetracyline derivatives such as demeclocycline. The compound may be selected from the group consisting of: (6R,12aR)-2,3,6,7,12,12a-Hexahydro-6-(5-benzofuranyl)-2-methyl-pyrazino[2′,1′:6,1 ]pyrido[3,4-b]indole-1,4-dione,

(6R,12aR)-2,3,6,7,12,12a-Hexahydro-6-(5-benzofuranyl)-pyrazino[2′,1′:6,1]pyrido[3,4-]indole-1,4-dione, (6R,12aR)-2,3,6,7,12,12a-Hexahydro-6-(5-benzofuranyl)-2-isopropyl-pyrazino[2′,1′:6,1]pyrido[3,4-b]indole-1,4-dione, (3S,6R,12aR)-2,3,6,7,12,12a-Hexahydro-6-(5-benzofuranyl)-3-methyl-pyrazino[2′,1′:6,1]pyrido[3,4-b]indole-1,4-dione, and

(3S,6R,12aR)-2,3,6,7,12,12a-Hexahydro-6-(5-benzofuranyl)-2,3-dimethyl-pyrazino[2′,1′: 6,1]pyrido[3,4-b]indole-1,4-dione, or from the group consisting of: KS-505, IC224, SCH 51866, IBMX and Dipyridamole. The disorder can be HD.

The invention also teaches the use of a composition which modulates PDE10 for treating a CAG repeat disorder comprising administering the composition to a subject in need of such treatment, and such use of the composition which modulates PDE10 for treating HD.

### BRIEF DESCRIPTION OF THE DRAWINGS

The foregoing features of the invention will be more readily understood by reference to the following detailed description, taken with reference to the accompanying drawings, in which:

FIG. 1 is a portion of an autoradiogram of the differential display reaction identifying PDE10A in mouse brain mRNA.

FIG. 2 is a northern blot confirming that PDE10A has a lower steady-state level of expression in the striatum of transgenic HD mice.

FIG. 3 is a nucleotide sequence of the differential display cDNA fragment of pPDE10A.

FIG. 4 shows the in situ hybridization of probe 1 to coronal and saggital brain sections of 10 week-old wild-type and HD mice.

FIG. 5 shows the in situ hybridization corresponding to spatial and temporal expression of PDE10A in brain sections of wild-type and HD mice over the period of time that the HD mice develop abnormal movements and postures.

FIG. 6 shows the in situ hybridization corresponding to expression of PDE10A in brain sections of one day old wild-type and HD mice.

FIG. 7 shows the in situ hybridization corresponding to distribution of the mRNA of PDE10A in mouse striatal neurons.

FIG. 8 is the in situ hybridization corresponding to mRNA distribution of the rat homologue of PDE10A in rat brain tissue.

FIG. 9 shows a Southern blot analysis of DNA from wild-type and transgenic HD mice hybridized to the pPDE10A cDNA probe.

FIG. 10 is a nucleotide sequence of cPDE10-1, and corresponds to SEQ ID NO. 1.

FIG. 11 is a restriction map of cPDE10-1.

FIG. 12 is a nucleotide sequence of cPDE10-2, and corresponds to SEQ ID NO. 2.

FIG. 13 is a restriction map of cPDE1O-2.

FIG. 14 is a schematic diagram showing the alignment of cPDE10-1 and -2 and the regions that are identical and unique between the two clones.

FIG. 15 is a nucleotide sequence of cPDE10A and RACEs, corresponding to SEQ ID NO. 11.

FIG. 16 is a map of PDE10A coding sequence and restriction sites.

FIG. 17 is a map of PDE10A coding sequence and features.

FIG. 18 is a restriction map of PDE10A.

FIG. 19 is a nucleotide sequence of cPDE10A and corresponds to SEQ ID NO. 12.

### DETAILED DESCRIPTION OF SPECIFIC EMBODIMENTS

The following illustrative explanations are provided to facilitate understanding of certain terms used frequently herein. The explanations are provided as a convenience and are not limitative of the invention. “Isolated” means altered “by the hand of man” from its natural state; i.e., that, if it occurs in nature, it has been changed or removed from its original environment, or both. As hereinbefore mentioned, the present inventors have identified and sequenced a DNA sequence encoding PDE10A. The DNA sequence is shown in the Sequence Listing as SEQ ID NO:1, NO:2 and NO:11.

It will be appreciated that the invention includes nucleotide or amino acid sequences which have substantial sequence homology with the nucleotide sequences shown in the Sequence Listing as SEQ ID NO:1, NO:2 and NO:11. The term “sequences having substantial sequence homology” means those nucleotide and amino acid sequences which have slight or inconsequential sequence variations from the sequences disclosed in the Sequence Listing as SEQ ID NO:1, NO:2 and NO:11; i.e. the homologous sequences function in substantially the same manner to produce substantially the same polypeptides as the actual sequences. The variations may be attributable to local mutations or structural modifications. It is expected that a sequence having 85-90% sequence homology with the DNA sequence of the invention will provide a functional PDE10 polypeptide.

As used herein, “PDE10A” comprises a polynucleotide sequence which is down regulated in the course of CAG repeat disorders selected from the group consisting of: (a) a sequence comprising SEQ ID NO:1; (b) a sequence comprising SEQ ID NO:2; (c) a sequence comprising SEQ ID NO:11; (d) a sequence comprising nucleotides 257 to 2596 of SEQ ID NO:11; (e) a sequence which is at least 90% homologous with a sequence of (a), (b), (c) or (d), and; (f) a sequence which hybridizes to (a), (b), (c) or (d) under stringent conditions. In an embodiment, the isolated polynucleotide segment is cDNA. The invention also teaches an isolated polynucleotide segment, which retains substantially the same biological function or activity as the polynucleotide encoded by the polynucleotide sequence.

The polynucleotides of the present invention may be employed as research reagents and materials for discovery of treatments of and diagnostics for disease, particularly human disease, as further discussed herein.

Analysis of the complete nucleotide and amino acid sequences of the protein of the invention using the procedures of Sambrook et al., supra, have been used to determine the expressed region, initiation codon and untranslated sequences of the PDE10A gene. The transcription regulatory sequences of the gene are determined by analyzing fragments of the DNA for their ability to express a reporter gene such as the bacterial gene lacZ.

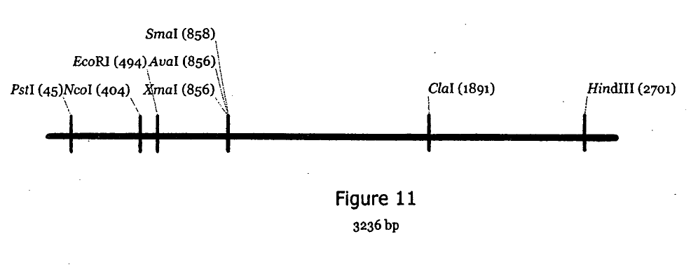

The nucleic acid molecules of the invention allow those skilled in the art to construct nucleotide probes for use in the detection of nucleotide sequences in biological materials. As shown in FIG. 11, 13, 15 and 16, a number of unique restriction sequences for restriction enzymes are incorporated in the nucleic acid molecule identified in the Sequence Listing as SEQ ID NO:1, NO:2 and NO:11, and these provide access to nucleotide sequences which code for polypeptides unique to the PDE10A polypeptide of the invention. Nucleotide sequences unique to PDE10A or isoforms thereof, can also be constructed by chemical synthesis and enzymatic ligation reactions carried out by procedures known in the art.

Detecting Presence of or Predisposition for CAG Repeat Disorders

This invention is also related to the use of the PDE10A polynucleotides to detect complementary polynucleotides as a diagnostic reagent. Detection of the level of expression of PDE10A in a eukaryote, particularly a mammal, and especially a human, will provide a method for diagnosis of a disease. Eukaryotes (herein also “individual(s)”), particularly mammals, and especially humans, exhibiting decreased levels of PDE10A may be detected by a variety of techniques. Nucleic acids for diagnosis may be obtained from an infected individual's cells and tissues, such as the striatum, nucleus accumbens and olfactory tubercule. RNA may be used directly for detection or may be amplified enzymatically by using PCR prior to analysis. As an example, PCR primers complementary to the nucleic acid encoding PDE10A can be used to identify and analyze PDE10A presence and/or expression. Using PCR, characterization of the level of PDE10A present in the individual may be made by comparative analysis.

The invention thus provides a process for detecting disease by using methods known in the art and methods described herein to detect decreased expression of PDE10 polynucleotide. For example, decreased expression of PDE10 polynucleotide can be measured using any on of the methods well known in the art for the quantification of polynucleotides, such as, for example, PCR, RT-PCR, DNAse protection, northern blotting and other hybridization methods. Thus, the present invention provides a method for detecting triplet-repeat disorders, and a method for detecting a genetic pre-disposition for triplet-repeat disorders and other disorders of the basal ganglia including schizophrenia, stroke, trauma, Parkinson's disease and dementia, such as Lewy Body disease. More generally, the present invention provides a method for detecting a genetic pre-disposition for neurological disorders characterized by progressive cell loss.

Drug Screening Assays

The invention also provides a method of screening compounds to identify modulators of PDE10A, i.e. those which enhance (agonist) or block (antagonist) the action of PDE10 polypeptides or polynucleotides, such as by interaction with PDE10-binding molecules. The identification of mutations in specific genes in inherited neurodegenerative disorders, combined with advances in the field of transgenic methods, provides those of skill in the art with the information necessary to further study human diseases. This is extraordinarily useful in modeling familial forms of triplet-repeat disorders and other disorders of the basal ganglia including schizophrenia, psychosis, stroke, trauma, Parkinson's disease and dementia. More generally, the present invention is useful for modeling neurological disorders characterized by progressive cell loss, as well as those involving acute cell loss, such as stroke and trauma.

For example, to screen for agonists or antagonists, a synthetic reaction mix, a cellular compartment, such as a membrane, cell envelope or cell wall, or a preparation of any thereof, may be prepared from a cell that expresses a molecule that binds PDE10. The preparation is incubated with labeled PDE10 in the absence or the presence of a candidate molecule which may be a PDE10 agonist or antagonist. The ability of the candidate molecule to bind the binding molecule is reflected in decreased binding of the labeled ligand.

PDE10-like effects of potential agonists and antagonists may by measured, for instance, by determining activity of a reporter system following interaction of the candidate molecule with a cell or appropriate cell preparation, and comparing the effect with that of PDE10 or molecules that elicit the same effects as PDE10. Reporter systems that may be useful in this regard include, but are not limited to, colorimetric labeled substrate converted into product, a reporter gene that is responsive to changes in PDE10 activity, and binding assays known in the art.

Another example of an assay for PDE10 antagonists is a competitive assay that combines PDE10 and a potential antagonist with membrane-bound PDE10-binding molecules, recombinant PDE10 binding molecules, natural substrates or ligands, or substrate or ligand mimetics, under appropriate conditions for a competitive inhibition assay. PDE10 can be labeled, such as by radioactivity or a colorimetric compound, such that the number of PDE10 molecules bound to a binding molecule or converted to product can be determined accurately to assess the effectiveness of the potential antagonist.

Potential antagonists include small organic molecules, peptides, polypeptides and antibodies that bind to a polynucleotide or polypeptide of the invention and thereby inhibit or extinguish its activity. Potential antagonists also may be small organic molecules, a peptide, a polypeptide such as a closely related protein or antibody that binds the same sites on a binding molecule, such as a binding molecule, without inducing PDE10-induced activities, thereby preventing the action of PDE10 by excluding PDE10 from binding.

Potential antagonists include a small molecule which binds to and occupies the binding site of the polypeptide thereby preventing binding to cellular binding molecules, such that normal biological activity is prevented. Examples of small molecules include but are not limited to small organic molecules, peptides or peptide-like molecules. Other potential antagonists include antisense molecules. Potential antagonists include compounds related to and derivatives of PDE10.

Developing modulators of the biological activities of specific PDEs requires differentiating PDE isozymes present in a particular assay preparation. The classical enzymological approach of isolating PDEs from natural tissue sources and studying each new isozyme may be used. Another approach has been to identify assay conditions which might favor the contribution of one isozyme and minimize the contribution of others in a preparation. Still another approach has been the separation of PDEs by immunological means. Each of the foregoing approaches for differentiating PDE isozymes is time consuming. As a result many attempts to develop selective PDE modulators have been performed with preparations containing more than one isozyme. Moreover, PDE preparations from natural tissue sources are susceptible to limited proteolysis and may contain mixtures of active proteolytic products that have different kinetic, regulatory and physiological properties than the full length PDEs.

Recombinant PDE10 polypeptide products greatly facilitate the development of new and specific PDE10 modulators. The need for purification of an isozyme can be avoided by expressing it recombinantly in a host cell that lacks endogenous phosphodiesterase activity (e.g., yeast strain YKS45 deposited as ATCC 74225). Once a compound that modulates the activity of PDE10 is discovered, its selectivity can be evaluated by comparing its activity on PDE10 to its activity on other PDE10 isozymes. Thus, the combination of the recombinant PDE10 products of the invention with other recombinant PDE10 products in a series of independent assays provides a system for developing selective modulators of PDE10. Selective modulators may include, for example, antibodies and other proteins or peptides which specifically bind to PDE10 or PDE10 nucleic acid, oligonucleotides which specifically bind to PDE10 (see Patent Cooperation Treaty International Publication No. WO93/05182 published Mar. 18, 1993 which describes methods for selecting oligonucleotides which selectively bind to target biomolecules) or PDE10 nucleic acid (e.g., antisense oligonucleotides) and other non-peptide natural or synthetic compounds which specifically bind to PDE10 or PDE10 nucleic acid. Mutant forms ofPDE10 which may have altered enzymatic activity or altered localization in a cell are also contemplated. Crystallization of recombinant PDE10 alone and bound to a modulator, analysis of atomic structure by X-ray crystallography, and computer modelling of those structures are methods useful for designing and optimizing non-peptide selective modulators. See, for example, Erickson et al., Ann. Rep. Med. Chem., 27: 271-289 (1992) for a general review of structure-based drug design.

Targets for the development of selective modulators include, for example: (1) the regions of PDE10 which contact other proteins and/or localize PDE10 within a cell, (2) the regions of PDE10 which bind substrate, (3) the allosteric cGMP-binding site(s) of PDE10, (4) the metal-binding regions of PDE10, (5) the phosphorylation site(s) of PDE10 and (6) the regions of PDE10 which are involved in dimerization of PDE10 subunits.

Thus, the present invention provides a method for screening and selecting compounds which modulate PDE10A and are potential therapeutic agents for treating psychosis, schizophrenia, obsessive-compulsive disorders, and other diseases and disorders such as stroke, trauma, Parkinson's disease, dementia, including Lewy Body disease, and Huntington's disease. More generally, the present invention provides a method for screening and selecting compounds which promote or inhibit neurological disorders characterized by progressive cell loss, as well as those involving acute cell loss, such as stroke and trauma.

The selected antagonists and agonists may be administered, for instance, to inhibit progressive and acute neurological disorders, such as psychosis, schizophrenia, obsessive-compulsive disorder, Huntington's disease, Parkinson's disease, dementia, for example Lewy Body disease, or stroke or trauma.

Antagonists and agonists and other compounds of the present invention may be employed alone or in conjunction with other compounds, such as therapeutic compounds. The pharmaceutical compositions may be administered in any effective, convenient manner including, for instance, administration by direct microinjection into the affected area, or by intravenous or other routes. These compositions of the present invention may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of antagonists or agonists of the invention and a pharmaceutically acceptable carrier or excipient. Such carriers may include, but are not limited to, saline, buffered saline, dextrose, water, glycerol, ethanol and combinations thereof. The formulation is prepared to suit the mode of administration.

### EXAMPLES

The present invention is further described by the following examples. These examples, while illustrating certain specific aspects of the invention, do not portray the limitations or circumscribe the scope of the disclosed invention.

### Example 1

### Isolation of PDE10A

Wild-type (B6CBAF1) and HD transgenic [B6CBA-TgN(Hdexon1)62Gpb] mice (Jackson Laboratories) and adult Sprague-Dawley rats (250-300 g; Charles River Laboratories) and were used in this study. The genotype of the mice was determined by PCR amplification of a 100 bp region of the integrated human HD exon 1 transgene using primers corresponding to nts 3340-3459 (5′-AGG GCT GTC AAT CAT GCT GG-3′) and nts 3836-3855 (5′-AAA CTC ACG GTC GGT GCA GC-3′) of clone E4.1 of the human HD gene (Accession number L34020). PCR conditions used are described in Mangiarini et al.(1996). DNA was extracted from a tail clip and an ear punch from each mouse used in this study. Both samples were subjected to PCR genotype analysis. For in situ hybridization analysis, the animals were anesthetized with >100 mg/kg sodium pentobarbital, decapitated, the brains removed and stored at −70° C. prior to sectioning. For RNA isolation, animals were anesthetized, decapitated and the striatum and cortex were excised and stored in liquid nitrogen prior to RNA extraction. Animal care was given according to protocols approved by Dalhousie University and the Canadian Council of Animal Care.

Differential display was used to identify novel mDNA or previously described mDNA whose relative expression levels are altered as a result of the presence of the transgene. Using differential display, the mRNA populations derived from the striatum of 10 week old wild type were compared with age-matched R6/2 transgenic mice. Differential display has been used extensively (>750 references) since its development to identify changes in gene expression in cells and in tissues including brain. Perhaps the most important finding was the demonstration that differential display can be used to isolate genes differentially expressed in inbred strains of mice. The power of differential display is that the sequence information obtained can be directly related to the experimental paradigm. Moreover, such sequence information includes sufficient information to identify transcripts and can then lead to experiments that reveal function of the cognate protein in the experimental model.

DNA sequence information of potentially differentially expressed cDNA can be used to generate oligonucleotide probes for in situ hybridization to define the anatomical and temporal patterns of expression of specific transcripts. This technique is especially useful to study changes in steady-state levels of mRNA in heterogeneous tissue such as brain. Brain tissue can be micro-dissected. This enabled the present inventors to reduce the requirement for tissue, and hence compare the mRNA populations derived from individual animals for each experimental group.

Thus RT-PCR was used to identify differences in the patterns of gene expression between the striatum of wild-type and transgenic mice that were hemizygous for the 5′ UTR, exon 1 and part of intron 1 of the human Huntingon's Disease gene. Total cellular RNA was isolated from the striatum and cortex of three 10 week-old wild-type and three 10 week-old R6/2 HD mice and used as the template to generate single-stranded cDNA. Total cellular RNA from each animal and tissue was purified using Trizol reagent (Gibco BRL) and the manufacture's protocol. 10 g aliquots of total RNA were treated with RQ1 DNAse-free DNAse (Promega) in the presence of DNAsin (Promega) DNAse inhibitor to remove trace genomic DNA and then converted to single-stranded cDNA. The primers and conditions for PCR amplification follow those of the Delta™ RNA fingerprinting manual (Clontech).

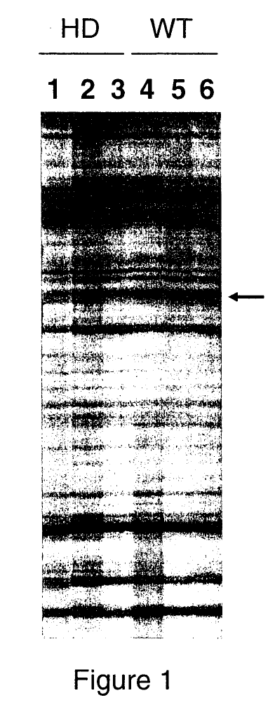

The cDNA was then used as the substrate for PCR reactions using 57 differential display primer combinations. The radio-labelled PCR products were fractionated on a denaturing acrylamide sequencing gels using a Genomyx LR sequencing apparatus, transferred to 3MM filter paper and dried. The dried acrylamide gels were exposed to autoradiography film (BioMax MR™) overnight. After fractionating the radio-labelled PCR products on denaturing acrylamide gels, it was found that the overwhelming majority of the approximately 18,000 PCR products screened were common to both the wild-type and HD mice (data not shown). One PCR product, amplified using the primers P7 (5′-ATT AAC CCT CAC TAA ATG CTG TAT G-3′) and T6 (5′-CAT TAT GCT GAG TGA TAT CTT TTT TTT TCG-3′) of approximately 500 bp, was observed in each of three samples derived from the striatum of wild-type mice (FIG. 1). This 500 bp band was absent from the samples derived from the striatum of the HD mice (FIG. 1) and was absent from each of the samples derived from the cortical tissue (data not shown).

FIG. 1 shows the Down-regulated in Huntington's Disease (PDE10A) transcript, identified by differential display RT PCR. A band of approximately 500 bp (arrow) was amplified from cDNA made form 10 week-old wild-type but not 10 week-old HD striatal tissue. Total RNA from individual animals (numbered 1-6) was used as the substrate for the generation of single-stranded cDNA. Animals 1, 2 and 3 were transgenic HD mice. Animals 4, 5 and 6 were wild-type mice.

### Example 2

### Cloning of PDE10A

The 500 bp band, designate PDE10Apcr, was excised from the dried gel and rehydrated in 40 μL of H₂O for 10 min at room temperature. The eluted DNA was subjected to PCR re-amplification using the P7 and T6 primers, rTaq polymerase (Pharmacia) and the following conditions: 60″ @ 94° C., 19×(30″ @ 94° C., 30″ @ 58° C., 120″ @ 68° C.+4″ per cycle), 7′ @ 68° C. The PCR reaction was subjected to agarose gel electrophoresis and the 500 bp band was removed from the gel, extracted from the agarose using the Qiagen gel extraction protocol and cloned into the vector, pGem-T using standard methods. Plasmid DNA was isolated from selected transformants using Qiagen spin columns. The resultant clone was named pPDE10A.

### Example 3

### Identification of PDE10A

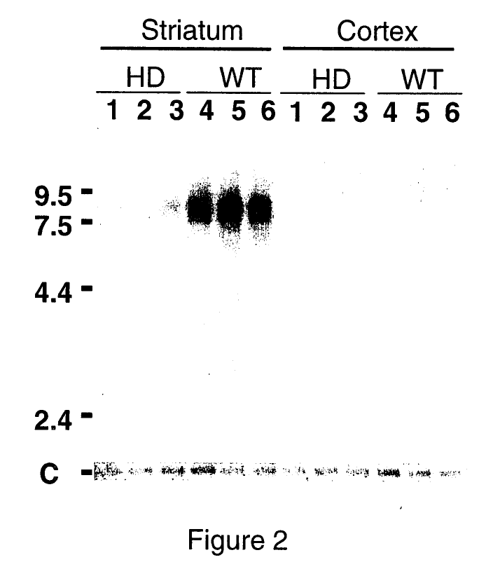

The cloned insert of pPDEIOA was radio-labelled and used as a hybridization probe in northern blot analysis (FIG. 2). Northern blots of total RNA were prepared using the method described in Denovan-Wright et al. (1998) Mol Brain Res 55, 350-354, the contents of which are incorporated by reference. The 500 bp cloned insert of PDE10A was radio-labelled with [−³²P]dCTP (3000 Ci/mmol) using the Ready-to-Go dCTP beads (Pharmacia).

Northern blot hybridization, brain tissue preparation and in situ hybridization are described in Denovan-Wright et al. (1998). The 500 bp cloned insert of pPDE10A annealed to a transcript of approximately 9.5 kb in total RNA isolated from the striatum of ten week-old wild-type mice.

FIG. 2 demonstrates that PDE10A is expressed in the striatum but not the cortex of wild-type mice and the steady-state levels of PDE10A are reduced in 10 week old transgenic HD mice. The differential expression of PDE10A in HD mice was confirmed by northern blot analysis. The cloned insert of pPDE10A was radio-labelled and used as a hybridization probe in northern blot analysis. The northern blot was prepared by size-fractionating total RNA from the striatum and cortex of three individual 10 week-old HD (1, 2 and 3) and wild-type (4, 5 and 6) mice. Following the hybridization of pPDE10A, the radio-label was removed and the blot was subsequently allowed to hybridize with a probe that detects constituitively expressed cyclophilin. The hybridization pattern of the cyclophilin probe is aligned below the northern blot demonstrating that equivalent amount of RNA were present in each lane. The relative mobility of RNA molecular weight standards (RNA ladder, Gibco BRL) are shown on the left of the northern blot.

The hybridization signal of pPDE10A was significantly lower in the RNA samples derived from the striatum of 10 week-old HD mice. No expression of the PDE10A mRNA was detected in the cortical RNA samples derived from either the wild-type or HD mice.

### Example 4

### Sequencing PDE10A

The sequence of the cloned differential display band, pPDE10A, was determined using M13 universal forward and reverse sequencing primers and the T7 sequencing kit (Pharmacia). The 484 bp cDNA fragment did not have sequence similarity to any Genbank entries.

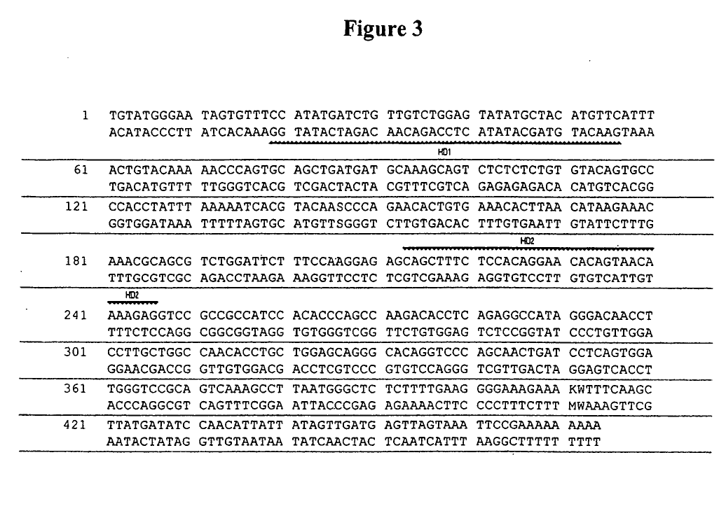

FIG. 3 shows the nucleotide sequence of the cloned PDE10A differential display product, pPDE10A. The position of the primers used to amplify the fragment are underlined and labelled. The nucleotide sequence and position of oligonucleotide probes 1 and 2 within the pPDE10A sequence are shown.

### Example 5

### Isolation and Characterization of cDNA PDE10A

In order to isolate PDE10A cDNA clones, oligonucleotide probes 1 and 2 were used in 5′ and 3′ Rapid Amplification of cDNA Ends (RACE) reactions using commercially prepared RACE-ready mouse striatal cDNA (Clontech). Several independent clones were isolated and those that contained the sequence of pPDE10A were selected for further analysis. Each of the 5′ RACE clones was identical in sequence over the length that the clones could be aligned. The difference in length between these clones is a result of termination of the original reverse-transcriptase reaction at different positions along the mRNA. No difference in size or sequence was detected between several 3′ RACE clones.

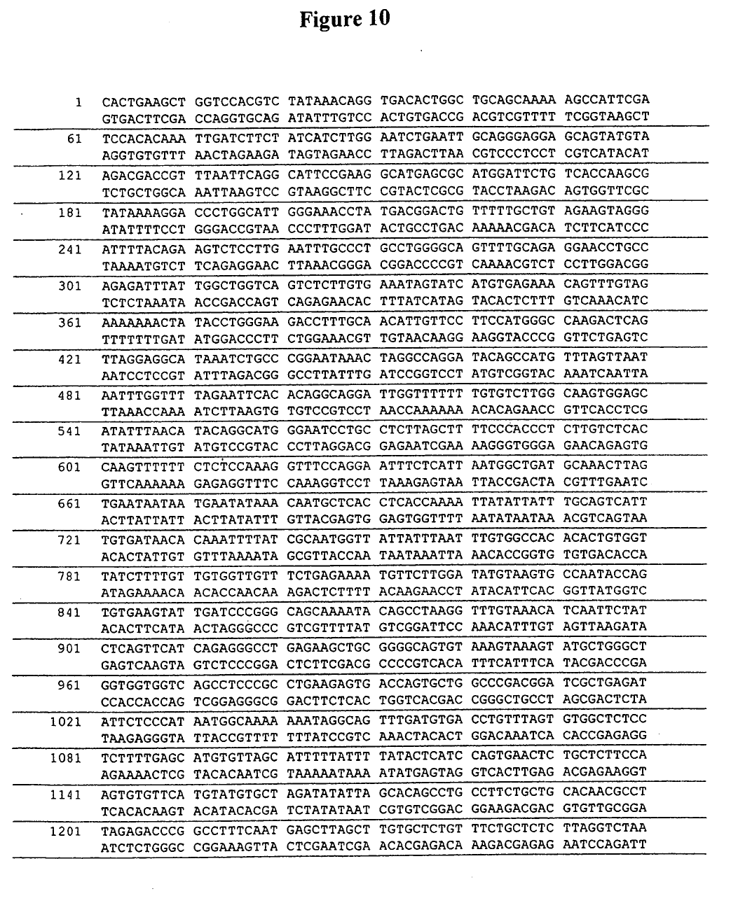

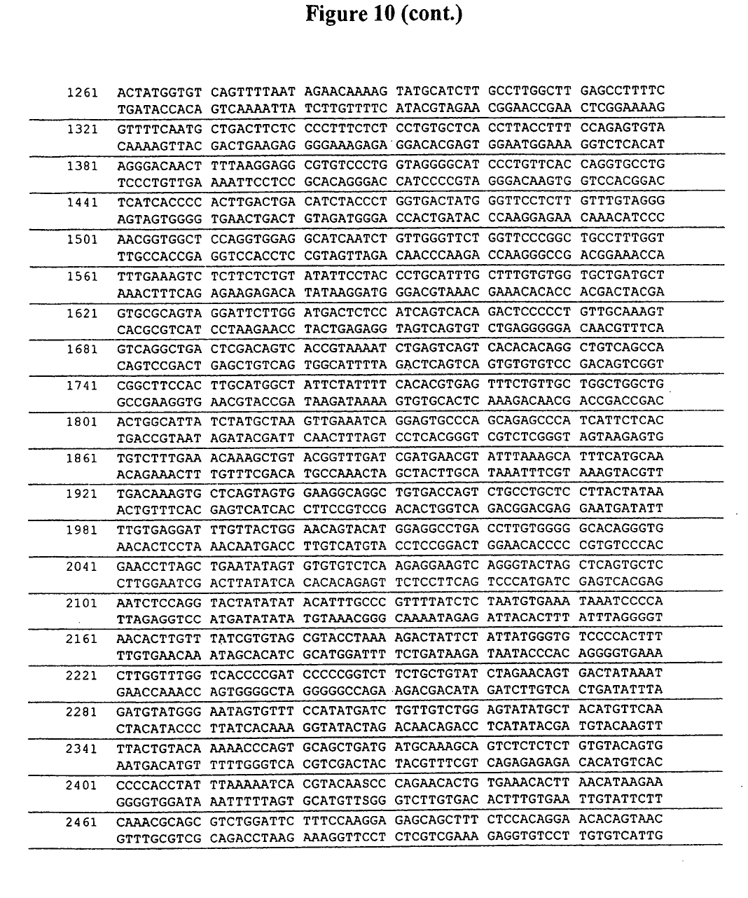

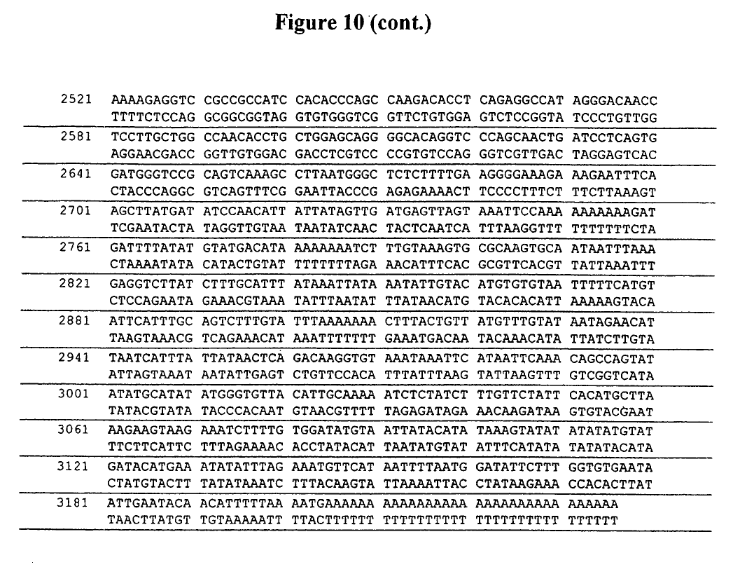

The longest 5′ RACE clone and one 3′ RACE clone were completely sequenced using internal primers. The present inventors were able to isolate a very short clone that extended the 5′ RACE clone using an internal primer (probe 3,5′-CTA TTT CAC AAG AGA CTG ACC AGC CAA TAA ATC TC-3′). The compiled sequence of the first PDE10A cDNA clone, named cPDE10A-1 is presented in FIG. 10. cPDE10A-1 is 3235 bp in length. The restriction map of cPDE10A-1 is shown in FIG. 11.

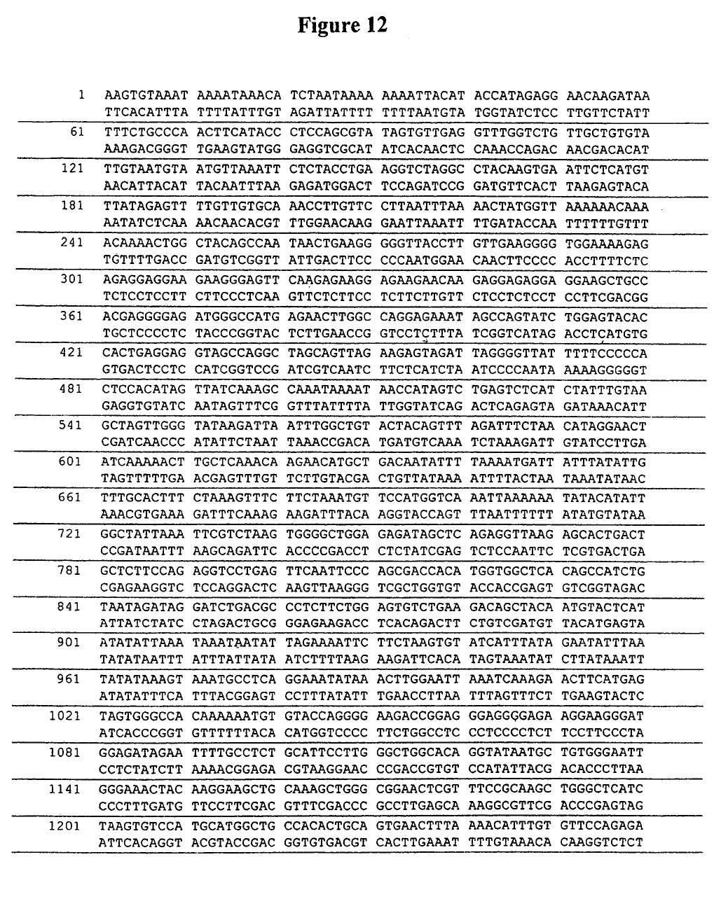

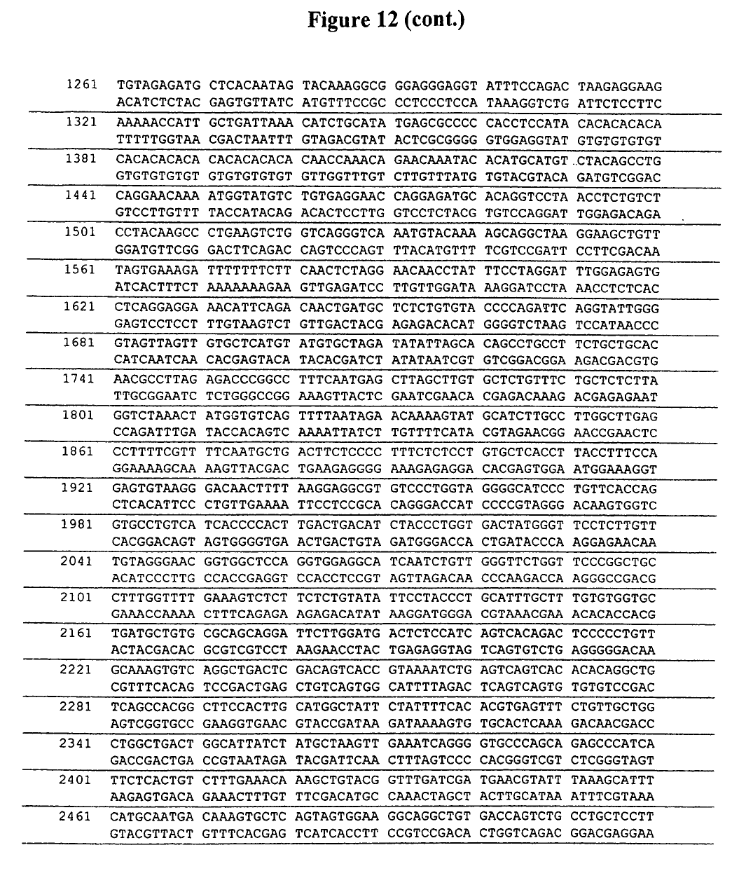

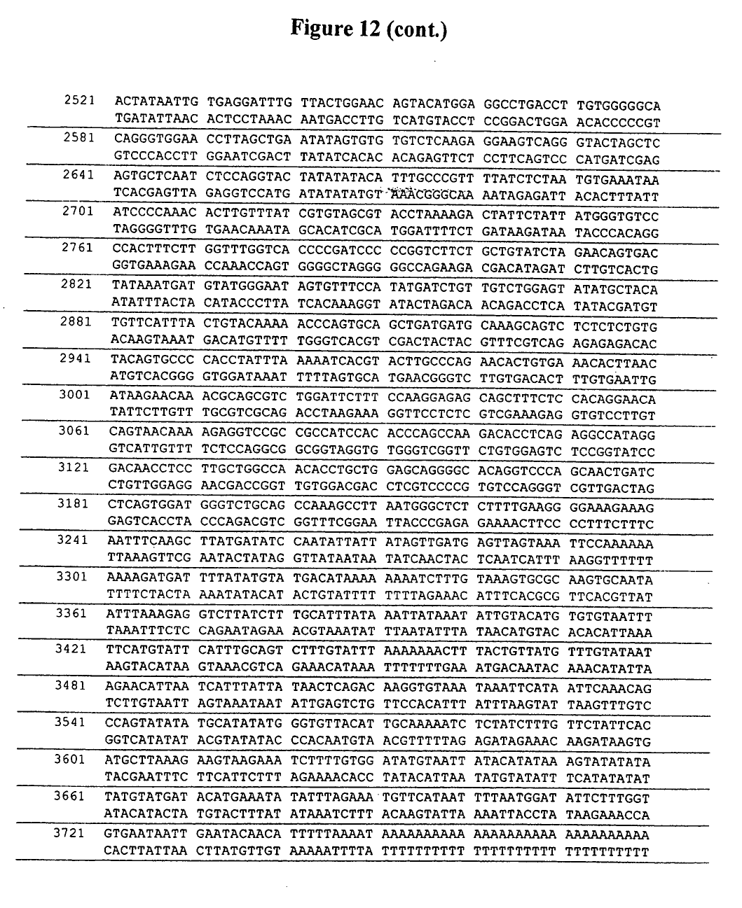

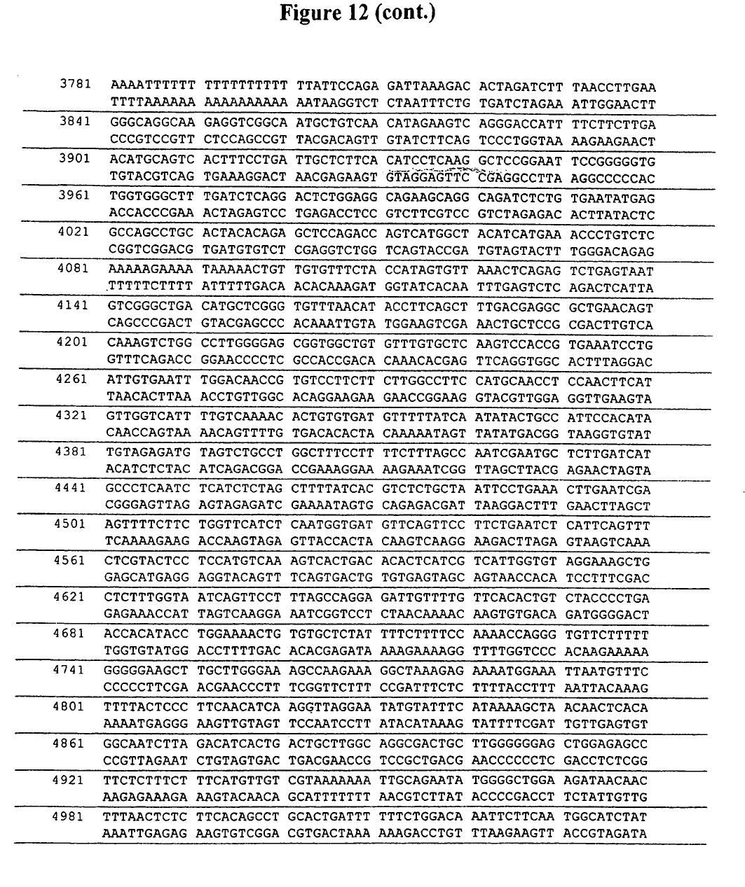

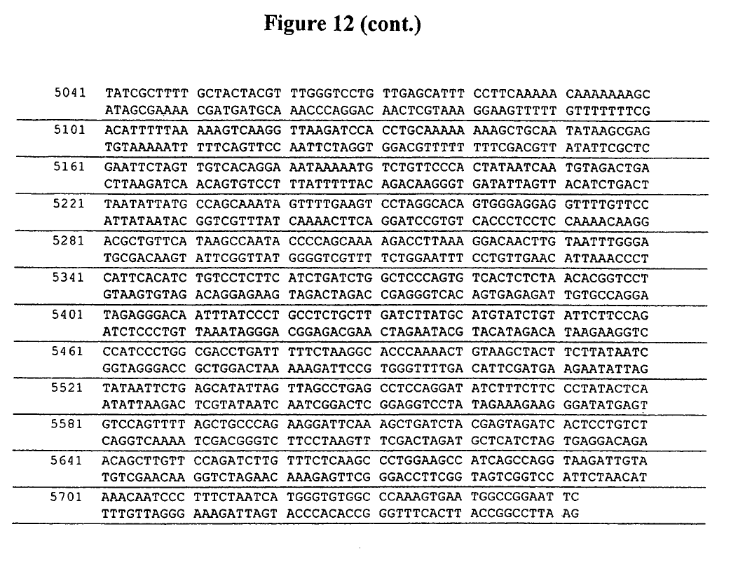

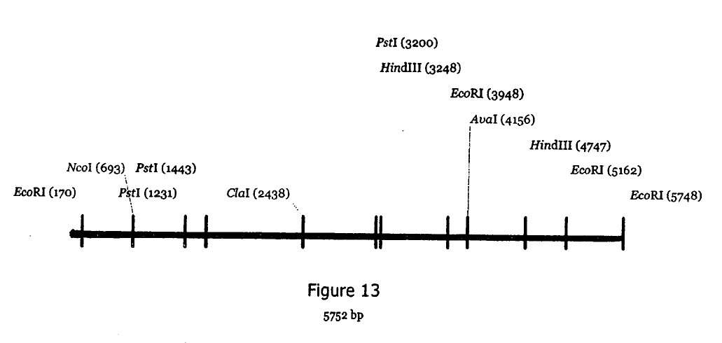

The mRNA that hybridized with pPDEI OA was approximately 9.5 kilobases in length. In order to obtain PDE10A cDNA clone that was larger than cPDE10-1, the present inventors screened a mouse brain cDNA library. Several clones were identified that hybridized with the pPDE10 probe. The sequence of the largest of these cDNA clones, cPDE10-2, was determined. The sequence (FIG. 12) was 5753 base pairs in length. The restriction map of cPDE10-2 is shown in FIG. 13.

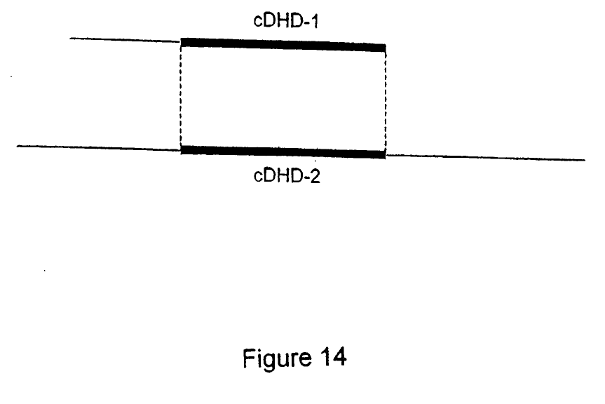

cPDE10-1 and cPDE10-2 share sequence identity over 2095 bp. However, the 5′ 1142 bp of cPDE10-1 and the 5′ 1689 bp of cPDE10-2 are unique to each clone. Clone cPDE10-2 extends 1969 bp in the 3′ direction compared to cPDE10-1. A schematic showing the regions of sequence identity and the unique sequences of cPDE10-1 and -2 are shown in FIG. 14.

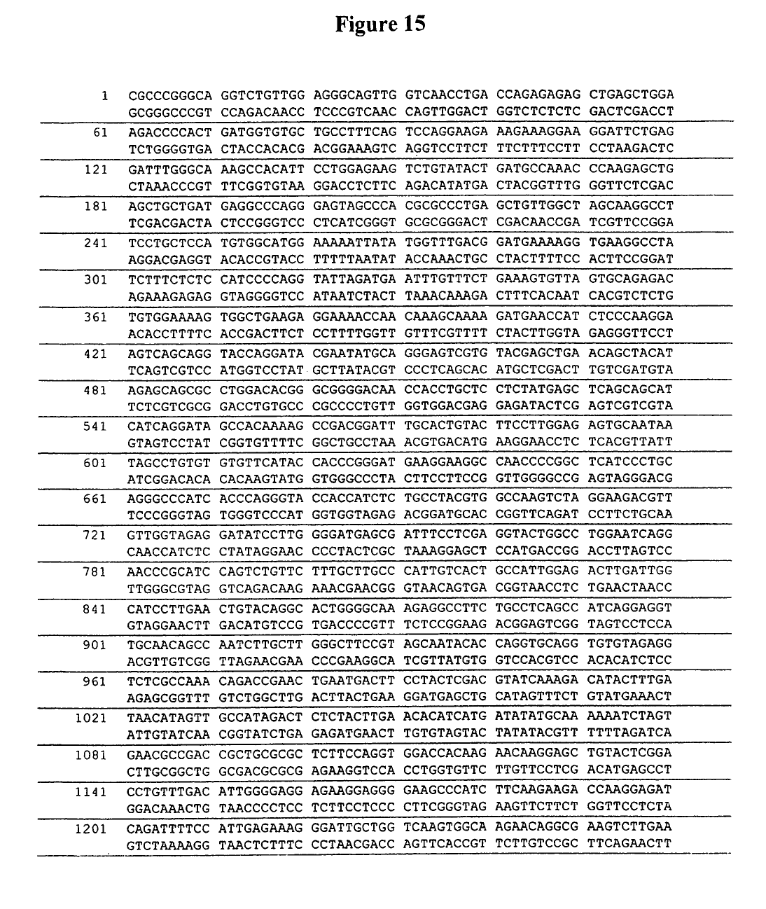

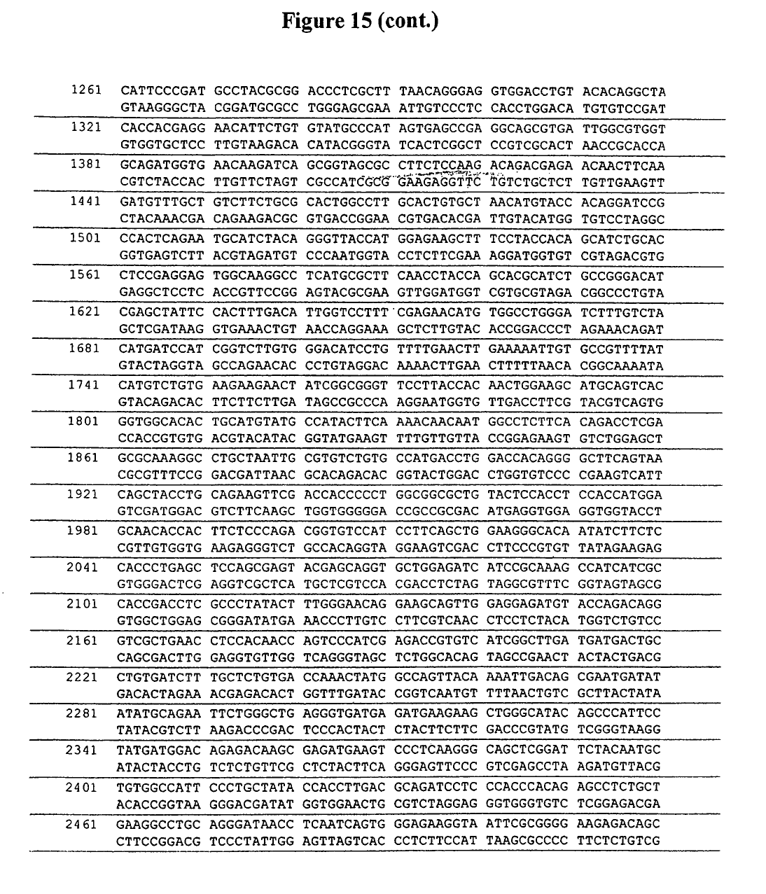

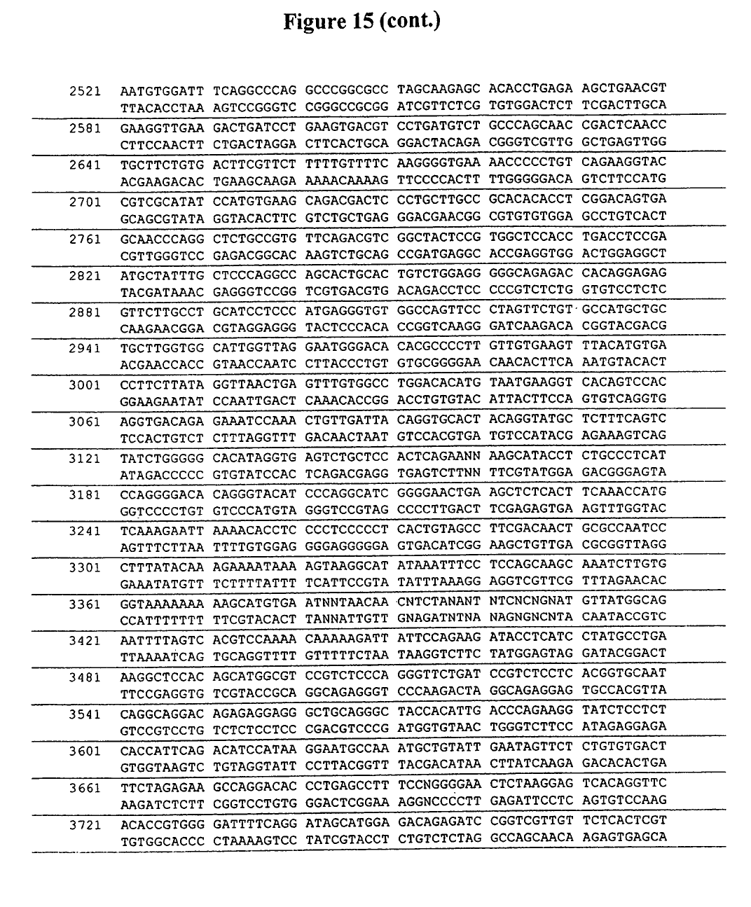

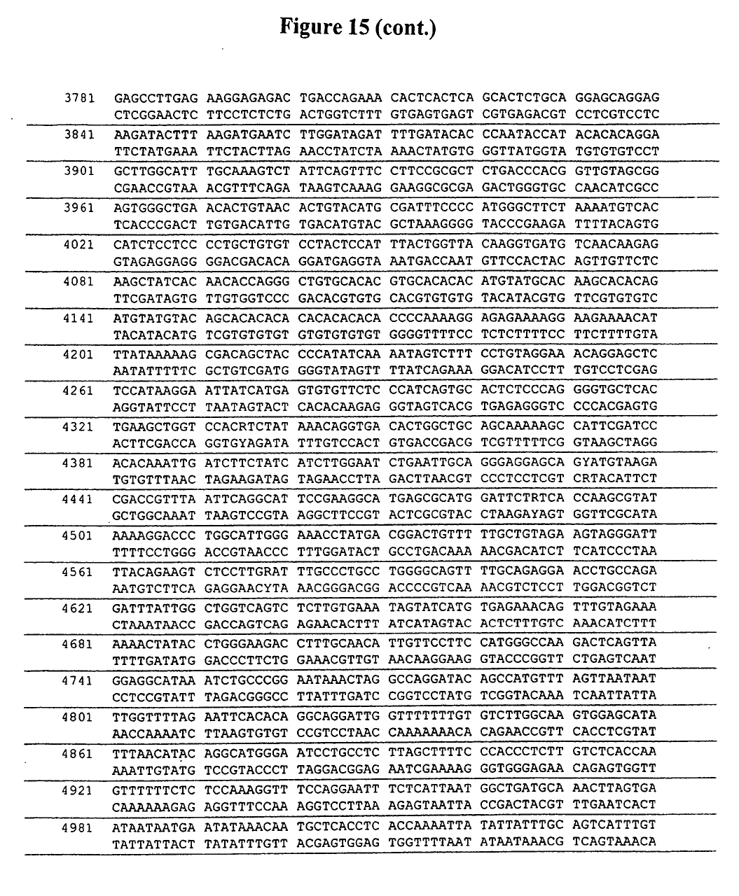

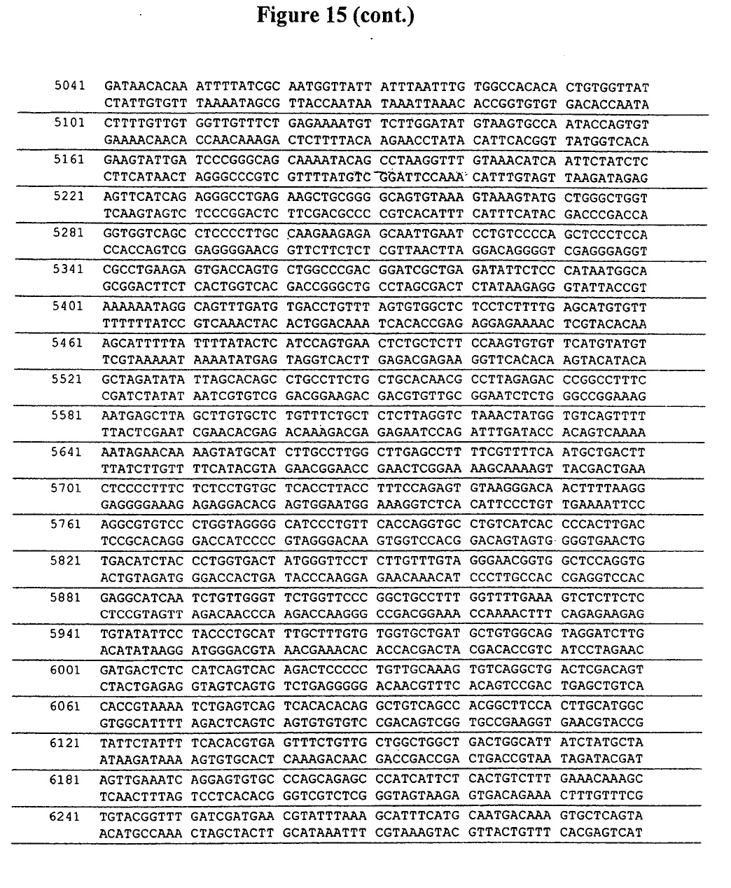

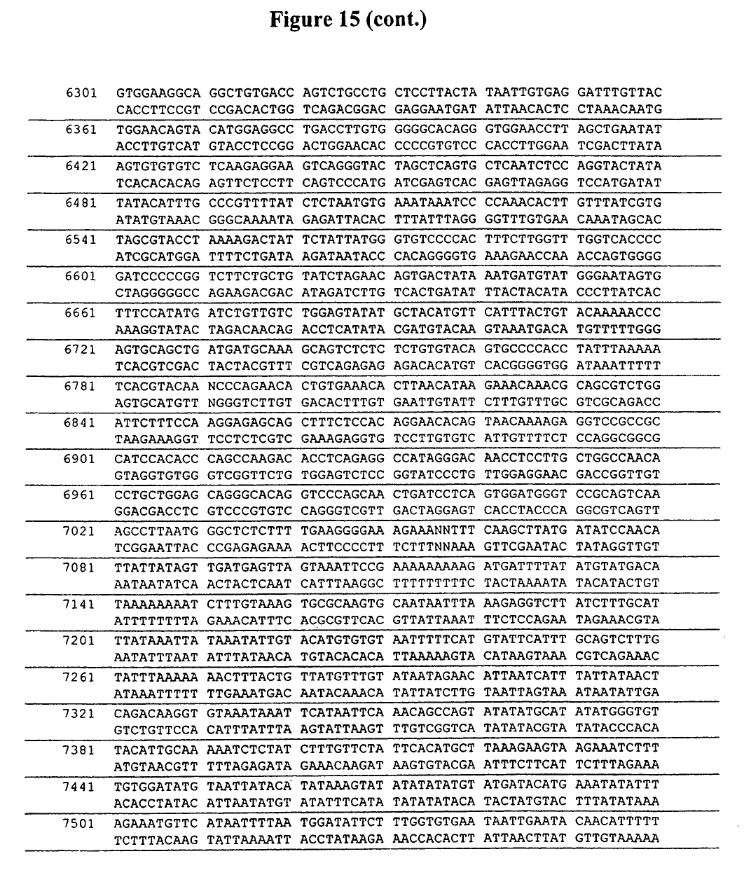

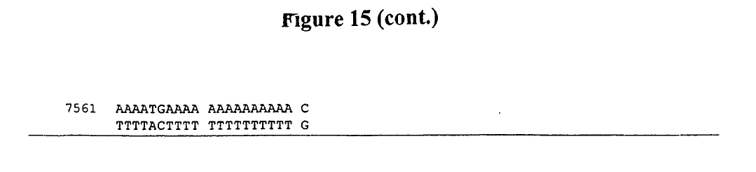

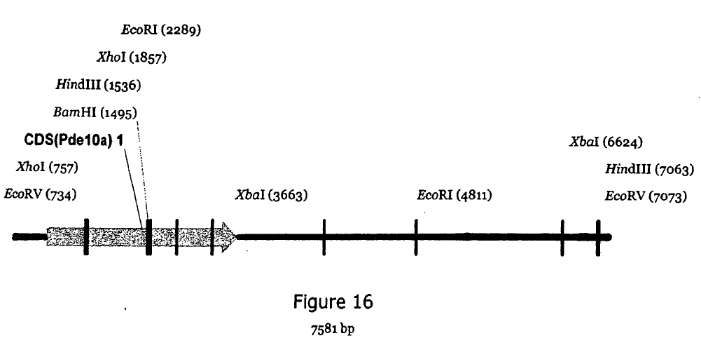

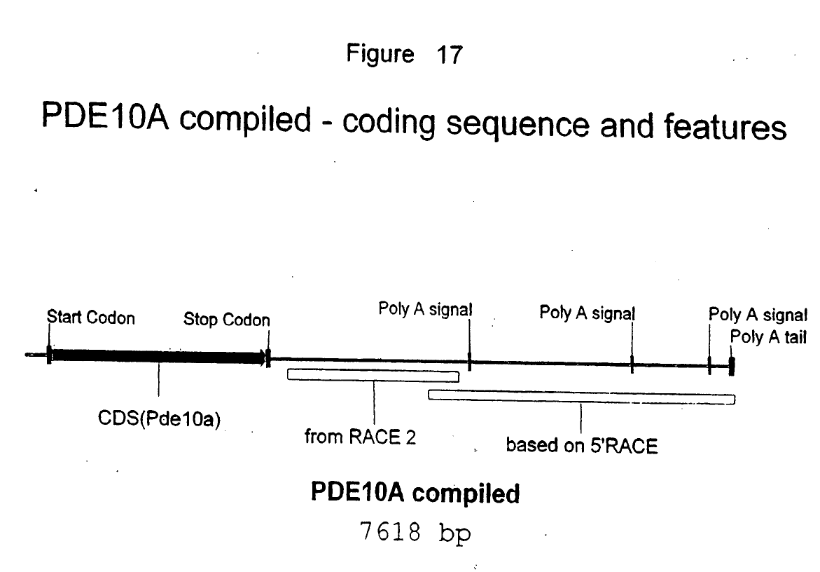

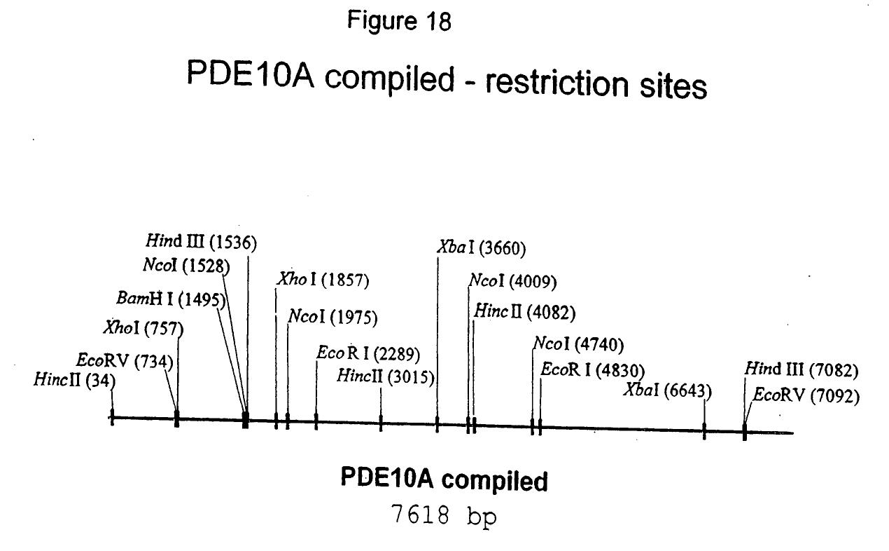

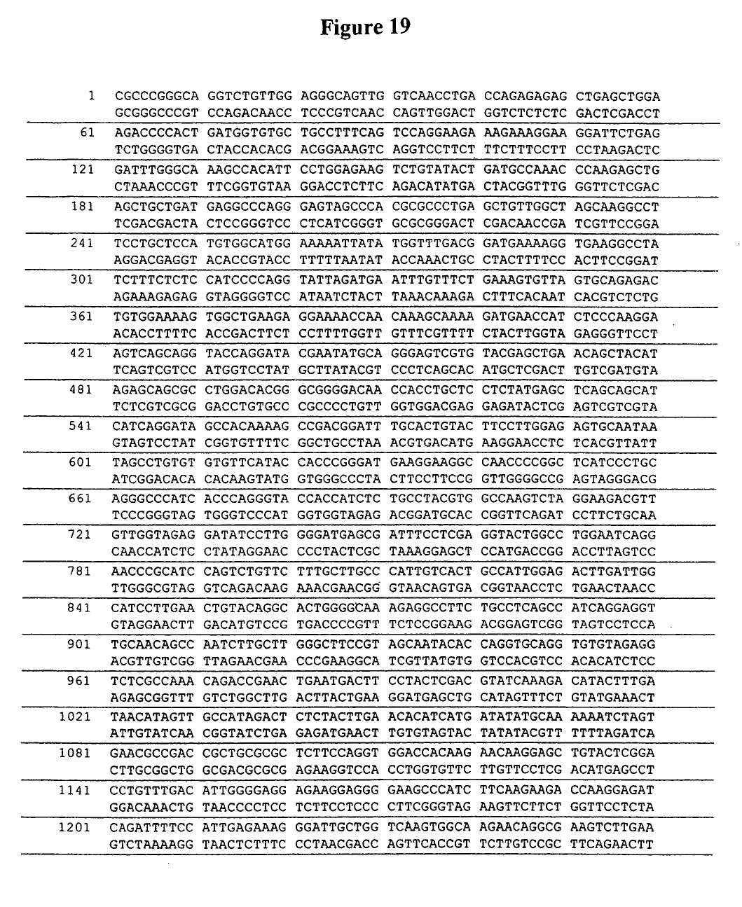

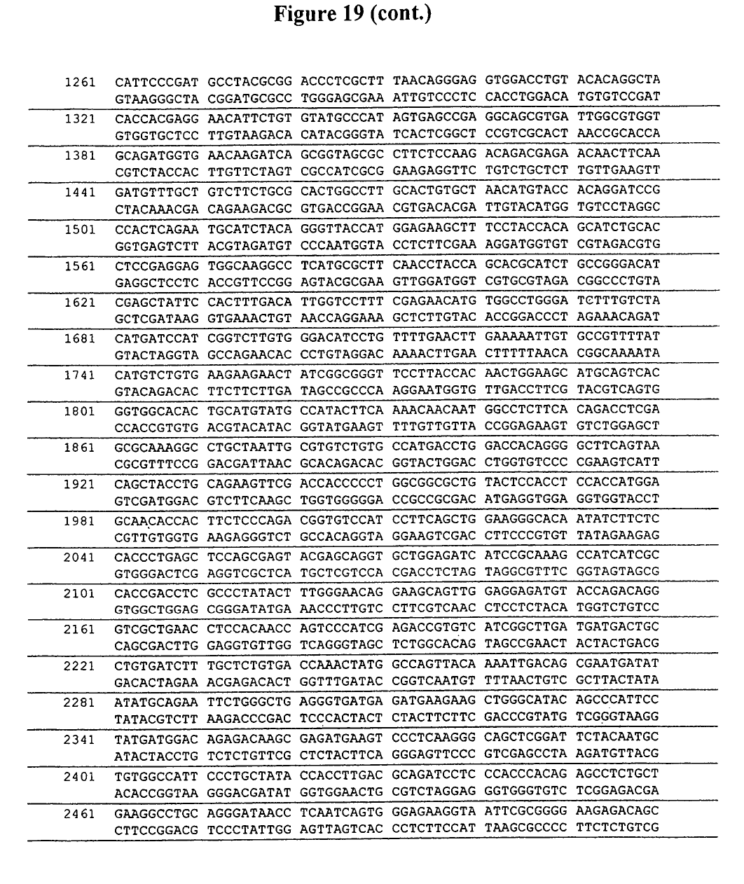

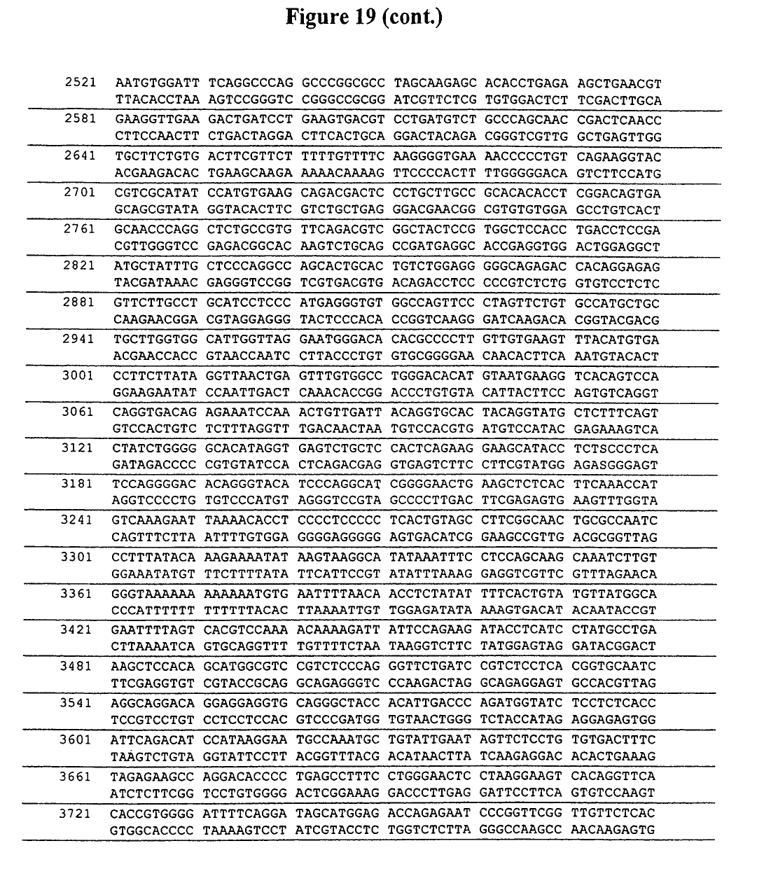

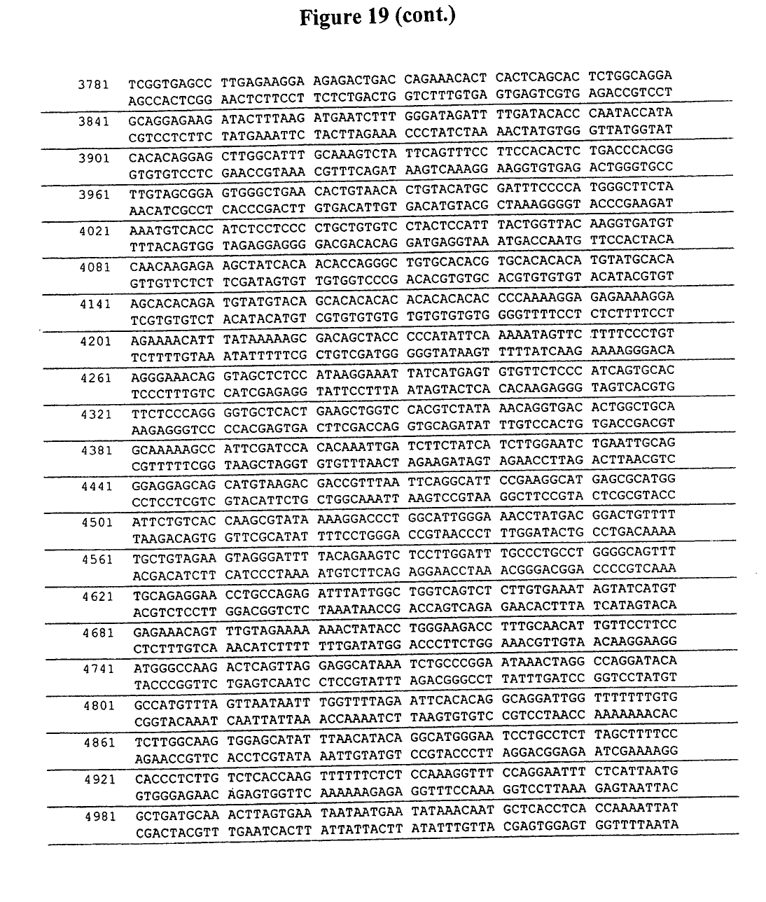

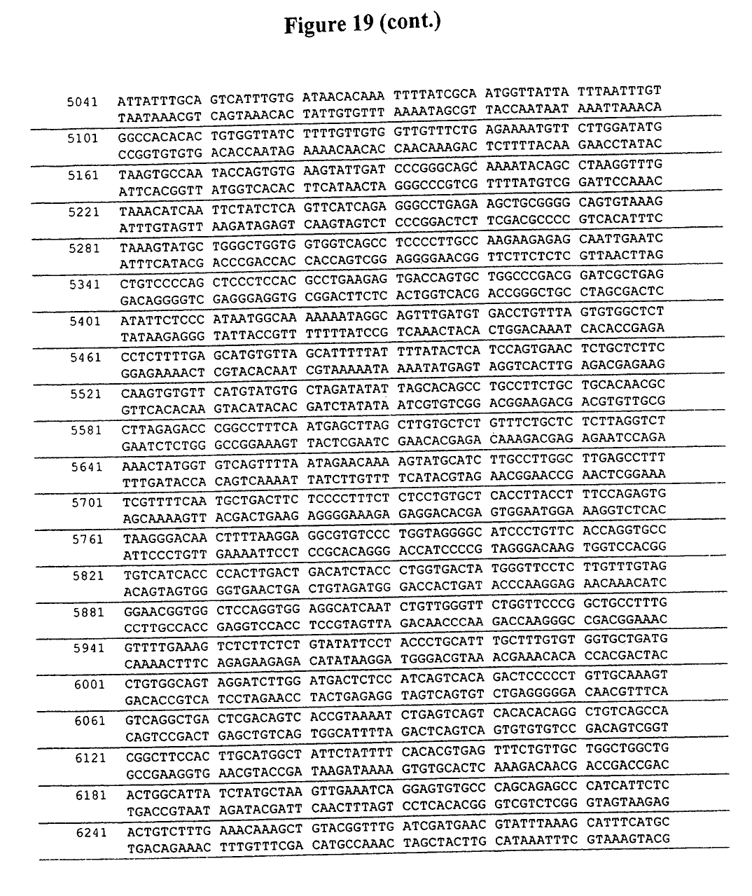

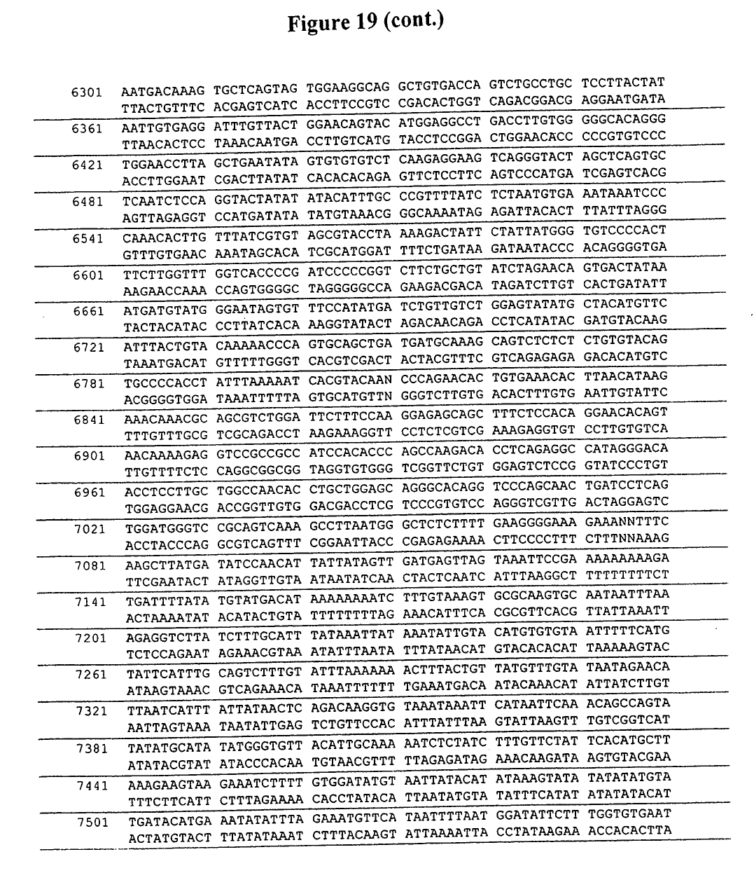

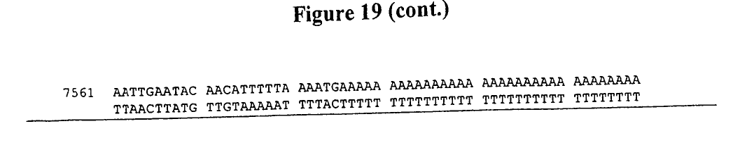

The compiled sequence of the mouse PDE10 cDNA clone, named cPDE10A, is presented in FIG. 15 with RACEs. A further sequence, without RACEs, is shown in FIG. 19. The coding sequence and restriction map of cPDE10A is shown in FIG. 16, and updated at FIG. 17. FIG. 18 is a restriction map of PDE10A. The coding region has a met initiator commencing at nucleotide 257, with a stop codon ending at nucleotide 2596.

PDE10A was found to have extremely high homology with human PDE10s identified by Loughney et al., WO99/42596, the contents of which are incorporated herein by reference.

### Example 6

### Localization of PDE10A in the Brain

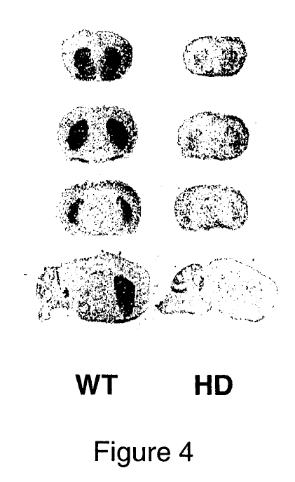

In order to identify the coding strand and to localize the transcript in the wild-type mouse brain, two oligonucleotide probes were designed (probe 1,5′-GAA CAT GTA GCA TAT ACT CCA GAC AAC AGA TCA TAT GG -3′; probe 2,5′-CAG CTT CTC CAC AGG AAC ACA GTA ACA AAG AG -3′) that were complementary to different regions and strands of the 484 bp pPDE10A clone. These oligonucleotides were used for in situ hybridization analysis. Using high stringency post in situ hybridization washes (2×30′ in 1×SSC @ 58° C., 4×15′ in 1×SSC @ 58° C., 4×15′ in 0.5×SSC @ 58° C., 4×15′ in 0.25×SSC @ 58° C.), it was found that oligonucleotide probe 1 annealed with mRNA in the striatum, nucleus accumbens and olfactory tubercule of ten week-old wild-type mice (FIG. 4). The hybridization signal was significantly reduced in the striatum, nucleus accumbens and olfactory tubercle of the 10 week-old HD mice (FIG. 4).

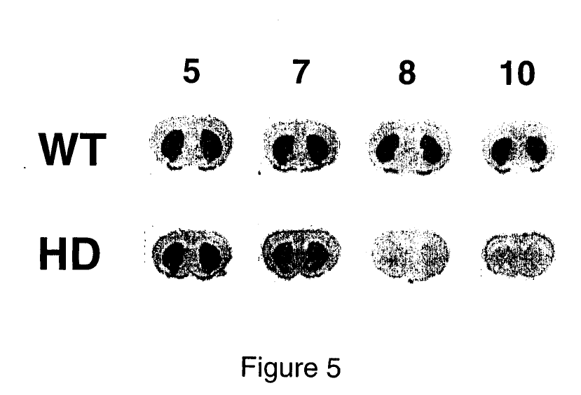

FIG. 5 shows in situ hybridization of probe 1 to coronal (top three sections) and saggital (bottom section) 10 week-old wild-type (WT) and HD mouse brain sections. Specific hybridization of the probe was observed in the striatum, nucleus accumbens and olfactory tubercle of wild-type mice. The top three sections represent the distribution of PDE10A throughout the rostral-caudal axis of the striatum.

The in situ hybridization results confirmed the northern blot analysis demonstrating, 1) that the expression of PDE10A mRNA was restricted to the striatum, nucleus accumbens and olfactory tubercle and 2) that the levels of PDE10A mRNA were decreased in HD mice compared to the wild-type. The probe did not anneal with mRNA in any other brain nuclei. No hybridization of oligonucleotide probe 2 was observed in any region of the brain in wild-type or HD mice (FIG. 3). Based on this hybridization, the coding strand, complementary to probe 1, of pPDE10A was defined.

### Example 7

### Characterization of PDE10

The in situ hybridization using oligonucleotide probe 1 demonstrated that PDE10A mRNA levels in the striatum , nucleus accumbens and olfactory tubercule were decreased in ten week- old HD mice. By ten weeks of age, the HD mice all showed motor symptoms including resting tremor and stereotypic involuntary movements. Moreover, these mice immediately clasped their feet together and curled into a tight ball when picked up by their tails.

As the phenotypic signs are progressive over a number of weeks, the present inventors examined whether the PDE1 OA transcript was ever expressed in the striatum of the HD mice or whether the steady-state levels of the transcript diminished in the striatum in a course that parallelled the development of the motor disorders. Wild-type and HD mice were sacrificed at 5, 7 and 8 weeks of age and their brains were prepared for in situ hybridization analysis using probe 1 (FIG. 5).

FIG. 5 shows the levels of PDE10A mRNA decrease in HD mice over the period of time that the HD mice develop abnormal movements and postures. In situ hybridization analysis of coronal and saggital sections of wild-type and HD mouse brain using oligonucleotide probe 1 which is complementary to the coding strand of PDE10A. At 5 weeks of age, before the development of motor symptoms, the HD mice express the PDE10A transcript in the same brain nuclei and at the same relative levels as wild-type mice. The steady-state level PDE10A decreases in the striatum, nucleus accumbens and olfactory tubercle from 5 to 10 weeks in the HD but not wild-type mice. By 9 weeks of age, the HD mice have abnormal movement and posture. The numbers refer to the age in weeks of the wild-type (WT) and Huntington's (HD) transgenic mice.

None of the mice at these ages had overt motor symptoms. Sections taken throughout the rostral-caudal axis of the striatum showed that PDE10A was expressed in the 5 week-old wild-type and HD mice. The relative hybridization of probe 1 did not change in 5, 7, 8 and 10 week-old wild-type mice. The intensity of the hybridization signal appeared to decrease in the striatum, nucleus accumbens and olfactory tubercle of HD mice from 5 to 10 weeks compared to their wild-type litter mates (FIG. 5).

The levels of PDE10A were significantly reduced by 8 weeks of age in the HD mice, using two in situ oligonucleotide probes, one complementary to the 3′ UTR, the second complementary to an internal portion of the coding region. The hybridization pattern observed in the wild-type and HD mice was the same for both the probes employed. This analysis demonstrated that there is a reduction in the complete PDE10A mRNA levels during the development of the HD phenotype and not that there was a differential reduction in the PDE10A coding region as compared to the extensive 3′ UTR. Moreover, in situ hybridization using the PDE10A-specific probe against neurologically normal and HD human brain tissue demonstrated that there was a decrease in PDE10A levels in human HD patients.

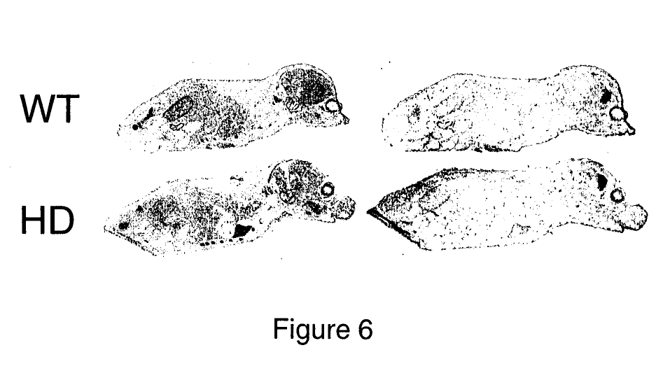

One day old wild-type and HD mice were frozen, sectioned on a cryostat and whole mouse sections were prepared for in situ hybridization using probe 1. The same high stringency post-hybridization washing conditions were employed for the one day-old mouse body sections as were used for the adult mouse brain sections. Parallel in situ hyridization experiments using the probe 2 were performed in order to determine the level of non-specific signal in the mouse sections. Probe 1 specifically annealed to the developing striatum (FIG. 6).

FIG. 6 demonstrates that PDE10A is expressed in the developing striatum of one day-old wild-type and HD mice. The sections on the left were subjected to in situ hybridization using probe 1. Following hybridization, the sections were counter-stained with cresyl violet to visualize the mouse organs. The signal outside the brain was non-specific as probe 2 and other unrelated control oligonucleotide probes all labelled these tissues.

There was no difference in the pattern of hybridization between the one day-old wild-type and HD mice demonstrating that PDE10A was expressed in the developing brain of both wild-type and HD mice.

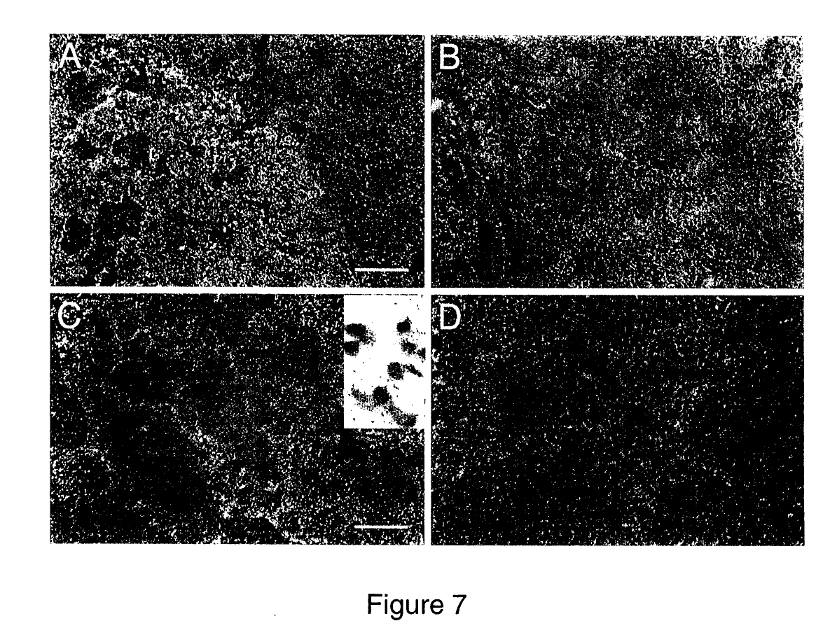

Following in situ hybridization, the sections were covered in autoradiographic emulsion, left in the dark to expose for 4 weeks and then developed and viewed under dark-field microscopy or, after counter-staining the sections with cresyl violet to visualize neuronal cell bodies, under bright-field microscopy. Silver grains were observed to be concentrated in the striatum of the wild-type mice. FIG. 7 shows emulsion autoradiography of mouse brain sections following in situ hybridization of probe 1 demonstrated that the PDE10A transcript is expressed in neurons. PDE10A is not homogeneously distributed throughout the mouse striatum. Dark field illumination of the sections after emulsion autoradiography showed that the silver grains were clustered in specific regions of the 10 week old wild-type mouse striatum (A and C). Sections from 10 week old HD mice subjected to identical in situ and emulsion autoradiographic conditions are shown in B and D. The photomicrographs shown in A and B were viewed using the 10× objective (bar represents 100 μm). The micrographs shown in C and D, were viewed under the 20× objective (bar represents 25 μm). The insert in panel C is a portion of the section in A and C counter-stained with cresyl violet to visualize the neurons, viewed using the 40× objective under bright filed illumination. Note the distribution of the silver grains over some, but not all, of the striatal neurons as well as being concentrated around clusters of neurons. It appeared that the silver grains were absent from fibre tracks within the striatum. It appeared that PDE10A mRNA was not confined to regions close to the nucleus but was dispersed in cellular processes.

Huntingtin with an expanded polyglutamine tract (htt-HD) is expressed in neurons of the brain and body throughout development and during the lifetime of HD patients. Transgenic HD mice express a portion of htt-HD and develop a phenotype with many of the symptoms of HD after a period of normal development and growth. Using differential display RT PCR, northern blot and in situ hybridization, we have demonstrated that PDE10A mRNA levels decline in the striatum of HD mice. This specific member of the PDE multigene family is highly expressed in the striatum and olfactory tubercle of mice and in the caudate and putamen of humans. The levels of PDE10A were the same in 5 week old wild-type and HD mice. PDE10A mRNA levels then began to decline and were almost undetectable in the striatum and olfactory tubercle by the time the mice reached 8 weeks of age. This time coincides with the onset of overt motor symptoms in the HD mice indicating that the loss of PDE10A in striatal neurons leads to dysfunction of the nuclei that control movement. The R6/2 mice develop the HD phenotype in the absence of cell death. The decrease in PDE10A mRNA, therefore, is not due to the loss of PDE10A-expressing cells but rather a change in steady-state RNA levels that occurs due to the expression of mutant huntingtin.

The particular isoform that decreases in HD is PDE10A. PDE10A has been cloned from human lung and fetal brain cDNA libraries. It appears that the presence of the expanded polyglutamine tract in huntingtin alters gene expression in the striatum, and that this is the mechanism by which only a small group of neurons in the striatum and cortex are rendered vulnerable to this ubiquitously expressed mutant protein.

### Example 8

### PDE10A is Highly Conserved Among Mammalian Species

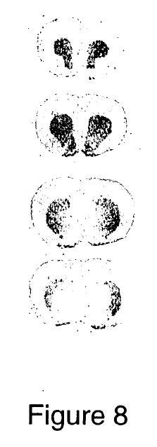

The oligonucleotide (probe 1) complementary to the coding strand of the PDE10A transcript, was also used as an in situ hybridization probe against coronal brain sections derived from adult rats. FIG. 8 shows in situ hybridization analysis of adult rat brain sections using oligonucleotide probe 1 complementary to the coding-strand of PDE10A revealed that the pattern of expression of PDE10A is the same in rats and mice. The hybridization conditions used to detect the rat homologue of PDE10A in rat brain tissue differed from those used to detect the transcript in mice only in that the stringency of the post-hybridization washes were reduced.

No hybridization was observed in the rat striatum using the post-hybridization washes employed following the in situ hybridization of mouse brain sections. However, when the stringency of the post-hybridization washes was lowered (2×60′ in 1×SSC @ 42° C., 2×60′ in 0.5×SSC @ 42° C., 2×60′ in 0.25×SSC @ room temperature), the PDE10A oligonucleotide probe specifically labelled the adult rat striatum, nucleus accumbens and olfactory tubercule in a pattern indistinguishable from that observed in mouse brain sections. It appears, therefore, that a transcript which shares nucleotide sequence and expression pattern is present in both mice and rats. The evolutionary conservation of PDE10A suggests that it is important for normal function of the basal ganglia.

By northern blot reports have demonstrated that PDE10A is expressed in human fetal brain. The homology between mouse and human PDE10A is extremely high (data not shown).

### Example 9

### Analysis of PDE10A in Genomic DNA

Because the transgenic mice employed in this study have a copy of the human HD 5′ UTR, exon 1 with expanded CAG repeat and 262 bp of the intron 1 that has been integrated into an undefined locus of the mouse genome, it was possible that the integration event disrupted the PDE10A gene preventing its expression in the HD mouse striatum. Genomic DNA was isolated from wild-type and HD mice and subjected to Southern blot analysis.

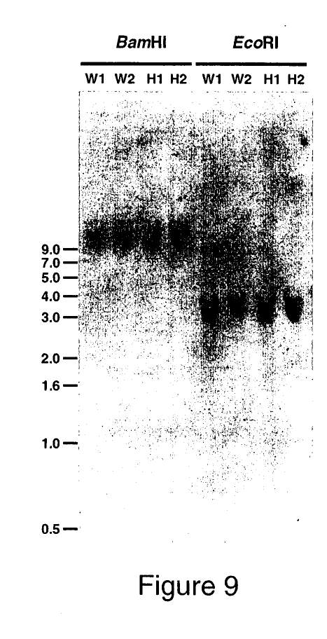

Genomic DNA was isolated from wild-type and HD mice and subjected to Southern blot analysis using pPDE10A as a hybridization probe. The size of the BamHI and EcoRI fragments that are present in the transgenic R6/2 line that correspond to the insertion of the human exon 1 gene fragment are 1.9 and 0.8 (BamHI) and 1.9 (EcoRI) kb. Analysis of the size of the fragments that hybridized with pPDE10A demonstrated that there was no difference in the size of the hybridizing fragments between the wild-type and HD mice. FIG. 9 shows the genomic DNA restriction fragments that hybridized with pPDE10A were the same in wild-type and HD mice. The size of the hybridizing BamHI and EcoRI fragments in each genomic DNA sample is approximately 8 kb and 3 kb, respectively. If the 1.9 kb SacI-EcoRI HD gene fragment integrated into the genome within the BamHI and EcoRI fragments that hybridized with the DHDM cDNA cloned insert, the sizes of the HD hybridizing bands would have been distinct from those of the wild-type. This Southern blot analysis indicates that the gene encoding PDE10A is present as a single-copy in the mouse genome. The numbers at the left of the blot are the relative mobility of molecular weight markers (1 kb ladder, BioRad).

The PDE10A cDNA has since been cloned using a bioinformatics search strategy involving screening of the expressed sequence tag (EST) database for novel PDE cDNA clones. Independently, the mouse PDE10A cDNA was identified after an EST search for novel PDEs with conserved cGMP binding domains. The rat isoforms of PDE10A and splice variants have also been described. Human, mouse and rat PDE10A splice variants differ in their 5′ untranslated and part of the 5′ coding region but are identical in the coding region when the various splice variants are compared within each species. The human, mouse and rat PDE10A coding regions contain 779, 779 and 794 amino acids, respectively, encoding a protein of approximately 88.5 kDa.

### Example 10

### Distribution of PDE10A

In mouse, PDE10A mRNA was detected in testis and to a much lesser extent in brain but not in heart, spleen, lung, liver, skeletal muscle, kidney, ovary, pancreas, smooth muscle, eye or in total RNA isolated from 7, 11, 15 or 17 day old embryo. This data agrees with the PDE10A mRNA pattern of distribution that we observed in wild-type and pre-symptomatic HD mice. In mice, two different size transcripts are detected in northern blots using the coding region as a probe. In mouse testis, the most abundant transcript is approximately 4 kb. A 9.5 kb transcript was also detected in mouse testis. It appears that the most abundant transcript in mouse brain is 9.5 k. Similarly, two sized PDE10A transcripts were observed in rats, however, it appears that, in rat, the 4 kb mRNA is expressed exclusively in testis while the 9.5 kb mRNA is expressed exclusively in brain. Within the brain, the rat PDE10A mRNA was expressed in striatum and olfactory tubercle and not cortex, cerebellum, hippocampus, midbrain or brainstem. In humans, PDE10A is expressed in the caudate, putamen and testis. As was observed in rodents, mRNAs of approximately 4 and 10 kb hybridized with the PDE10A probe. Again, it appears that, although both sized transcripts are present in brain and testis, the larger mRNA is predominant in the caudate and putamen and the smaller sized transcript is present in the testis. Each of the mouse, rat and human PDE10A sequences are not longer than 4 kb and span the coding region and parts of the 3′ UTR. The difference in abundance of the short and long transcript in the testis and brain, respectively, in all three species suggest that the 3′ UTR functions to provide transcript stability in the brain. As such, we present the complete sequence of the brain-specific transcript of PDE10A derived from mouse.

### Example 11

### Modulators of PDE10A Activity

Eight compounds were synthesized and tested for PDE10A activity using a scintillation proximity assay (SMA). The compounds, and their characteristics, are listed below:

### Compound NN101—1,2-dihydro-1-oxobenzofuro[2,3-c]pyridine-7-carboxylic acid

Molecular formula: C₁₂H₇NO₄
Structure: 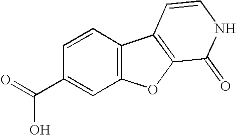
Melting point: Decomposed higher 320° C.
Solubility: Soluble in aqueous base, concentrated sulfuric acid, TFA, slightly soluble in DMSO, insoluble in acetic acid, THF, alcohol and ethyl acetate.
Characterization and Purity: ¹H NMR, ¹³C NMR and TLC pure
Toxicity: N/A
Storage: Room temperature
Stability: Solid at room temperature

### Compound NN111—1,2-dihydro-1-oxobenzofuro[2,3-c]pyridine-6-carboxylic acid

Molecular formula: C₁₂H₇NO₄
Structure: 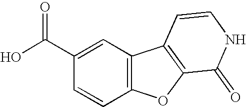
Melting point: Starting decomposed at 220° C.
Solubility: Soluble in aqueous base, concentrated sulfuric acid, and TFA, slightly soluble in DMSO and hot acetic acid, insoluble in THF, alcohol and ethyl acetate.
Characterization and Purity: ¹H NMR, ¹³C NMR and TLC pure
Toxicity: N/A
Storage: Room temperature
Stability: Solid at room temperature

### Compound NN113—(2E)-3-(1,2-dihydro-1-oxobenzofuro[2,3-c]pyridin-6-yl)acrylic acid

Molecular formula: C₁₄H₉NO₄
Structure: 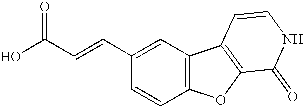
Melting point: Gradually decomposed at 264° C.
Solubility: Soluble in aqueous base, concentrated sulfuric acid, TFA, and DMSO, slightly soluble in hot acetic acid, insoluble in THF, alcohol and ethyl acetate.
Characterization and Purity: ¹H NMR, ¹³C NMR and TLC pure
Toxicity: N/A
Storage: Room temperature
Stability: Solid at room temperature

### Compound NN121—1,2-dihydro-1-oxobenzofuro[2,3-c]pyridine-8-carboxylic acid

Molecular formula: C₁₂H₇NO₄
Structure: 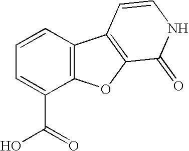
Melting point: Decomposed higher 300° C.
Solubility: Soluble in aqueous base, concentrated sulfuric acid, and TFA, slightly soluble in DMSO and hot acetic acid, insoluble in THF, alcohol and ethyl acetate.
Characterization and Purity: ¹H NMR, ¹³C NMR and TLC pure
Toxicity: N/A
Storage: Room temperature
Stability: Solid at room temperature

### Compound NN201—(Z)-5-((1H-indol-3-yl)methylene)-2-thiooxazolidin-4-one

Molecular formula: C₁₂H₈N₂O₂S
Structure: 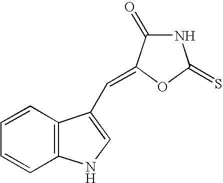
Melting point: Decomposed higher 280° C.
Solubility: very soluble in DMSO and DMF, soluble in THF
Characterization and Purity: ¹H NMR, ¹³C NMR and TLC pure
Toxicity: N/A
Storage: Room temperature
Stability: Solid at room temperature

### Compound NN202—(Z)-5-((1H-indol-3-yl)methylene)oxazolidine-2,4-dione

Molecular formula: C₁₂H₈N₂O₃
Structure: 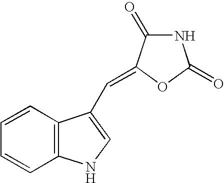
Melting point: Decomposed >280° C.
Solubility: very soluble in DMSO and DMF, soluble in THF, ethyl acetate
Characterization and Purity: ¹H NMR, ¹³C NMR and TLC pure
Toxicity: N/A
Storage: Room temperature
Stability: Solid at room temperature

### Compound NN203—(Z)-5-((1H-indol-3-yl)methylene)thiazolidine-2,4-dione

Molecular formula: C₁₂H₈N₂O₂S
Structure: 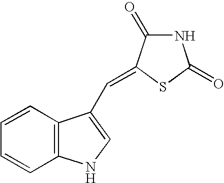
Melting point: Decomposed higher 320° C.
Solubility: very soluble in DMSO and DMF, slightly soluble in THF
Characterization and Purity: ¹H NMR, ¹³C NMR and TLC pure
Toxicity: N/A
Storage: Room temperature
Stability: Solid at room temperature

### Compound NN204—(Z)-5-((1H-indol-3-yl)methylene)-2-thiothiazolidin-4-one

Molecular formula: C₁₂H₈N₂OS₂
Structure: 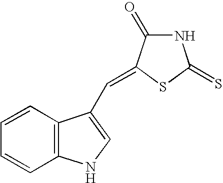
Melting point: Decomposed higher 300° C.
Solubility: very soluble in DMSO and DMF, soluble in THF
Characterization and Purity: ¹H NMR, ¹³C NMR and TLC pure
Toxicity: N/A
Storage: Room temperature
Stability: Solid at room temperature

### Example 12

### Characterization of PDE10A Modulators

The effects of the eight new compounds on PDE10A were characterized. Compoundsx categorized under general Formula I include NN101, NN111, NN113 and NN121. Compounds categorized under general Formula II include NN201, NN202, NN203, and NN204.

All eight compounds were designed, synthesized, and tested for purity. The structure and purity of these compounds were characterized by ¹H NMR, ¹³C, NMR and TLC. The solubility of the compounds was characterized in aqueous base, concentrated sulfuric acid, TFA, DMSO, hot acetic acid, THF, alcohol and ethyl acetate. The melting point of each compound was also characterized. All four compounds are a solid at room temperature and are stable at room temperature. Specific details as they relate to each individual compound are given below.

The PDE10A inhibitory activity for these compounds was characterized by scintillation proximity assay (SPA) and it was found that four of these compounds (NN101, NN121, NN111 and NN113) potently inhibit PDE10A activity.

Further studies were performed using the SMA assay to characterize the dose-response effect of each the four 100 series compounds on PDE10A activity. Results are reported in CPM (counts per minute) versus log concentration of the compound. All four Ki values are reported below; substrate concentration was used at ⅓ Km using a Km value for recombinant PDE10A activity of 0.16 μM.

NN101—SMA derived Ki value=123 μM

NN113—SMA derived Ki value=2.23 μM

NN111—SMA derived Ki value=38.9 μM

NN121—SMA derived Ki=323 μM

Recent studies support that knocking-out PDE10A has profound effects on striatal function and that this most likely occurs through modulation of glutamatergic neurotransmission (Neuropharmacology (2006) 51, 374-385). As mentioned, all four organic 100 series compounds (NN111, NN121, NN101, NN113) possess significant inhibitory effects on PDE10A activity. These initial results suggest that these compounds are candidates for selective inhibition of PDE10A. Additional studies will more completely characterize the biological potential of these compounds. Moreover, these results provide evidence that these compounds are useful as a new therapeutic class of drugs for the treatment of psychosis and schizophrenia, and for the novel application and treatment of psychotic symptoms associated with such disorders.

### Example 13

### Selective Modulators of PDE10A Activity

The catalytic domain of PDE10A is most similar in amino acid sequence to PDE5A, PDE2A, PDE6B and PDE6A. These members of the PDE family each contain a cGMP binding sequence that is not observed in other PDE family members. The non-catalytic cGMP binding sites (GAF) domains found in PDE2, 5 and 6 are also found in PDE10. At least for PDE2, this site acts as an allosteric activator for cAMP hydrolytic activity. The GAF domain of PDE10A binds other small molecules that act as allosteric activators. PDE10A is a cAMP and cAMP-inhibited cGMP PDE.

Attenuation of the production of cAMP, may ameliorate the symptoms of HD and positively affect gene expression. Pharmaceutically acceptable modulators of cAMP include quinpirole, alloxan, miconazole nitrate, MDL-12330A, and tetracyline derivatives such as demeclocycline and minocycline.

Compounds which are potent and selective modulators of cGMP-specific PDE, and are useful in a variety of therapeutic areas are taught by Daugan et al, U.S. Pat. No. 5,981,527, PCT publication No. WO 00/15639 to Icos Corporation and PCT publication No. WO 00/15228 to Icos Corporation, which are incorporated herein by reference. Such compounds include, for example:

(6R,12aR)-2,3,6,7,12,12a-Hexahydro-6-(5-benzofuranyl)-2-methyl-pyrazino[2′, 1′:6,1]pyrido[3,4-b]indole-1,4-dione,

(6R,12aR)-2,3,6,7,12,12a-Hexahydro-6-(5-benzofuranyl)-pyrazino[2′,1′:6,1]pyrido[3,4-]indole-1,4-dione,

(6R,12aR)-2,3,6,7,12,12a-Hexahydro-6-(5-benzofuranyl)-2-isopropyl-pyrazino[2′,1′:6,1]pyrido[3,4-b]indole-1,4-dione,

(3S,6R,12aR)-2,3,6,7,12,12a-Hexahydro-6-(5-benzofuranyl)-3-methyl-pyrazino[2′,1′:6,1]pyrido[3,4-b]indole-1,4-dione, and

(3S,6R,12aR)-2,3,6,7,12,12a-Hexahydro-6-(5-benzofuranyl)-2,3-dimethyl-pyrazino[2′,1′:6,1]pyrido[3,4-b]indole-1,4-dione.

PDE1B1 is expressed throughout the brain and is most abundant in the striatum, nucleus accumbens and olfactory tubercle (Polli and Kincaid, 1994; Yan et al., 1994). PDE1B is a cGMP, Ca/calmodulin-dependent PDE. Therefore, PDE1B and 10A are both expressed in the majority, but not all, striatal neurons and, it is likely that both genes are co-expressed in a subset of striatal projection neurons. Selective inhibitors for PDE1 include KS-505, IC224, and SCH 51866. Of these inhibitors, it appears that SCH 51866 has a ten-fold higher Km for PDE1 than PDE10. The non-specific PDE inhibitor IBMX is a potent inhibitor of PDE10A. Dipyridarnole and SCH51866 had the highest potency of inhibitors tested on PDE10A activity. Dipyridamole was considered to be a PDE5- and PDE6-specific inhibitor, however, the Km for dipyridamole is 10 times higher for PDE10A than the other PDEs. Selective inhibitors of PDE5, 2, 3 and 4 had much greater IC50 for PDE10.

### Example 14

### Modulating Activity of PDE10A Using cGMP-PDE Activity

Cyclic nucleotide phosphodiesterases (PDEs) are a superfamily of ubiquitously expressed isozymes that hydrolyze cyclic nucleotides adenosine 3′,5′-cyclic monophosphate (cAMP) and guanosine 3′,5′-cyclic monophosphate (cGMP). Cyclic nucleotides are the predominant second messengers that activate cellular signaling pathways. The concentration of intracellular cyclic nucleotides is dependent on their rate of synthesis by adenyl and guanyl synthase, the rate of efflux from the cell, and the rate of degradation. PDEs hydrolyze cAMP and cGMP limiting both the duration and amplitude of the cyclic nucleotide signal. PDEs are involved in cellular signaling by regulating levels of cAMP and cGMP, and effecting activation of their downstream mediators such as: protein kinase A (PKA), cyclic nucleotide-gated channel (CNG) and protein kinase G (PKG). Since many diseases have multifactorial origins, manipulating the multiple intracellular signaling pathways that involve cAMP/cGMP through PDEs may have profound therapeutic potential.

In mammals, PDEs are encoded by a large multigene family. The various PDE family members have tissue-specific patterns of expression. PDEs have also been described in Caenorhabditis, Drosophila, Dictyostelium, Saccharomyces, Candida and Vibrio species demonstrating that this enzyme has been conserved throughout evolution. In mammals, PDEs are encoded by at least 11 gene families, each composed of one or more genes. In addition, numerous splice variants of individual gene family members have been described. These splice variants alter the 5′ domain of the protein but share identical nucleotide binding and catalytic domains. The catalytic domain, found in the carboxy-terminus of the enzyme, is ˜275 amino acids and highly conserved in amino acid sequence in all PDEs. In total, it appears that there are ˜50 PDEs expressed within the mammalian body. Some PDEs are expressed in multiple tissues while others have a very limited tissue-specific distribution.

The PDE10 family was originally characterized in fetal lung and brain. PDE gene families differ with respect to their affinity for cAMP and cGMP and their dependence on calcium and calmodulin. Moreover, some PDEs are inhibited or activated by binding cyclic nucleotides to a non-hydrolytic site. For example, PDE2A has a lower Kₘ for cGMP than cAMP although it hydrolysed both nucleotides. The binding of cGMP to an allosteric activator site within PDE2 enhances the rate of catalysis of cAMP. PDE2 is, therefore, a cGMP-stimulated cGMP and cAMP phosphodiesterase. Conversely, the affinity of PDE4 for cAMP is much greater than for cGMP and PDE4 activity is not affected by cGMP or calmodulin. The differences in substrate preference, modulation of activity and tissue-specific patterns of expression suggest that subtle alterations in the relative levels of cAMP and cGMP mediated through the action of various PDEs lead to a wide range of responses to extracellular signals. Another isozyme, PDE10A, is particularly concentrated in the brain (caudate and putamen), testis and thyroid. This isoform is expected to have potential implications for the treatment of neurological disorders. There are currently no effective compounds designed specifically for the treatment of positive psychotic symptoms associated with diseases such as schizophrenia. Current therapies interfere with global cognitive function and possess many unwanted adverse side effects on the autonomic and central nervous system such as tardive dyskinesia. Evidence from Siuciak et al. (Neuropharmacology 51 (2006) 374-385) suggests that deletion of PDE10A maintains dopaminergic neurotransmission while modulating glutamatergic neurotransmission. Currently there are no selective inhibitors for the PDE10A family. Therefore, one embodiment of the invention provides dual-acting compounds of formula I or formula II that modulate PDE10A activity and also act as dopamine antagonists.

cGMP-PDE activity of compounds is measured using a one-step assay adapted from Wells at al. (Wells, J. N., Baird, C. E., Wu, Y. J. and Hardman, J. G., Biochim. Biophys. Acta 384:430 (1975)) and adopted by Beavo et al, U.S. Pat. No. 6,037,119. The reaction medium contains 50 mM Tris-HCl, pH 7.5, 5 mM Mg-acetate, 250 μg/mL 5′-Nucleotidase, 1 mM EGTA and 0.15 μM 8-[³H ]-cGMP. The enzyme used is a human recombinant PDE V (ICOS, Seattle U.S.A.).

Compounds of interest are dissolved in DMSO finally present at 2% in the assay. The incubation time was 30 minutes during which the total substrate conversion did not exceed 30%. The IC₅₀ values for the compounds examined are determined from concentration-response curves using typically concentrations ranging from 10 nM to 10 μM. Tests against other PDE enzymes using standard methodology also show compounds highly selective for the cGMP specific PDE enzyme.

Rat aortic smooth muscle cells (RSMC) are prepared according to Chamley et al. in Cell Tissue Res. 177:503-522 (1977) and used between the 10th and 25th passage at confluence in 24-well culture dishes. Culture media is aspirated and replaced with PBS (0.5 mL) containing the compound tested at the appropriate concentration. After 30 minutes at 37° C., particulates guanylate cyclase are stimulated by addition of ANF (100 nM) for 10 minutes. At the end of incubation, the medium is withdrawn and two extractions were performed by addition of 65% ethanol (0.25 mL). The two ethanolic extracts are pooled and evaporated until dryness, using a Speed-vat system. c-GMP was measured after acetylation by scintillation proximity immunoassay (AMERSHAM). The EC₅₀ values are expressed as the dose giving half of the stimulation at saturating concentrations.

Composition, Formulation, and Administration of Pharmaceutical Compositions

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally.grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to phosphoric, phosphonic, sulfonic, hydrochloric, sulfuric, acetic, lactic, tartaric, malic, succinic, and other acids which form suitable biocompatible salts.

Suitable routes of administration may, for example, include oral, rectal, transmucosal, transdermal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, the active agent may be administered to an individual as an injectable composition, for example as a sterile aqueous dispersion, preferably isotonic. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition. For administration to mammals, and particularly humans, it is expected that the daily dosage level of the active agent will be from 0.001 mg/kg to 10 mg/kg, typically around 0.01 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

The invention further provides diagnostic and pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

### Example 15

### Clinical use of New PDE10a Modulators

Patients showing symptoms of psychotic behavior, with or without additional clinical evidence such as abnormality of brain blood flow as determined by SPECT, MRI evidence for the presence of bilateral caudate atrophy, and/or global atrophy of the cerebrum and corpus callosum, are administered an initial effective dose (for example, from a range between 10, 25, 50, 100, 200, 500 and 1000 mg, or other dose determined to be effective for a given compound) of any one or more of the PDE10A inhibitors NN101, NN111, NN113, or NN121 twice daily for 7 days, followed by a 100% increase of the initial effective dosage in mg twice daily for 7 days, and finally a 200% increase of the initial effective dosage mg twice daily for 5 weeks or longer, as needed. In alternative treatments, the increases in dosage may be 50% and 100%, or 200% and 400%, or any other suitable increase, as desired or determined to be effective by a physician. The patient's psychotic symptoms (behavior) are compared to any of: baseline behavior for that patient in the absence of the PDE10A modulator; baseline behavior for that patient being treated with other anti-psychotic formulation(s); and/or to a control group of patients with similar clinical profiles and behavior being treated with other anti-psychotic drugs After 5 weeks of administration of the highest mg dosage twice daily of the PDE10A inhibitor, improvement in frequency and severity of psychotic episodes is compared to the baseline clinical global assessment made prior to the time of admission. Treatment may also be suspended for 7 days, or longer, as appropriate, and then re-instated, for evidence that observed effects are due to the administration of the PDE10A inhibitor and for evidence that the patient responds to treatment upon reinstatement of the treatment program. Improvements in clinical and behavioral symptoms suggest treatment should continue indefinitely.

### Example 16

### Clinical use of PDE10a Modulator

A 38 year-old female was admitted to hospital from a long-term care facility due to progressive deterioration of her physical and mental symptoms caused by Huntington's disease. The patient had been diagnosed with Huntington's disease at age 26. Prior to admission to the hospital, she had become increasingly aggressive and uncooperative. Moreover, there appeared to be an increase in the number of psychotic episodes. SPECT showed no abnormality of brain blood flow but MRI showed bilateral caudate atrophy as well as global atrophy of the cerebrum and corpus callosum.

The patient had been stable for a number of years on the antipsychotic haloperidol (3 mg/day). For the last two years, the haloperidol had been replaced by olanzapine (2.5-7.5 mg/day).

Minocycline, a tetracycline derivative, was administered at 50 mg twice daily for 7 days, followed by 100 mg twice daily for 7 days and finally 200 mg twice daily for 5 weeks. After 5 weeks of 200 mg twice daily minocycline administration, there was a mild improvement compared to the baseline clinical global assessment made at the time of admission. The minocycline treatment was suspended for 7 days. Due to a significant increase in the number of aggressive incidence and decrease in cooperativity, minocycline (200 mg twice daily) treatment was resumed. The patient responded within 3 days to the resumed minocycline-treatment with a return to mild-improvement compared to the baseline clinical global assessment made at the time of admission. Minocycline (200 mg twice daily) treatment will continue indefinitely. The improvement in behaviour and decrease in apparent psychosis has allowed for the transfer of the patient from the acute care facility back to long-term care.

While the present invention has been described in terms of specific embodiments, it is understood that variations and modifications will occur to those skilled in the art. Accordingly, only such limitations as appear in the appended claims should be placed on the invention.

1. A compound of formula I, 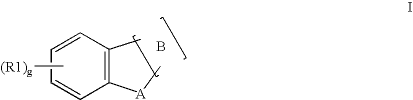
and pharmaceutically acceptable salts thereof, wherein
A is NR, O or S;
R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl;
g is 0, 1, 2, 3, or 4; and
B is a ring forming a fused ring system with the ring containing A and is selected from; 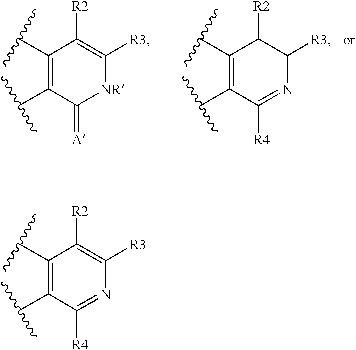
wherein A′ is as described above for A and NR′ is as described above for NR,
R1, R2, R3 and R4 are independently selected from:
(i) hydrogen, C₁ to C₅ alkyl, OH, NH₂, C₁ to C₅ alkylamino, di(C₁ to C₅
alkyl)amino, C₁ to C₅ alkylcarbonyl, C₁ to C₅ alkyloxycarbonyl, C₁ to C₅ alkylcarbonyloxy, carboxyl, C₁ to C₅ alkyl phosphonate, C₁ to C₅ alkenyl phosphonate, C₁ to C₅ alkyl phosphate, C₁ to C₅ alkenyl phosphate, C₁ to C₅ alkyl sulfonate, C₁ to C₅ alkenyl sulfonate, halo, halo(C₁ to C₅)alkyl, amino(C₁ to C₅)alkyl, hydroxyl(C₁ to C₅)alkyl, (C₁ to C₅)alkoxyl) C₁ to C₅ alkyl, NO₂, C₁ to C₅ alkylthio, SO₃H, PO₄, PO₃H, NH₄, C₂ to C₅ alkenyl, C₂ to C₅ alkenyloxy, C₂ to C₄ alkenylamino, di(C₂ to C₅ alkenylcarbonyl), C₂ to C5₄ alkenyloxycarbonyl, C₂ to C₄ alkylcarbonyloxy, halo(C₂ to C₅)alkynyl, amino(C₂ to C₅)alkenyl, hydroxy(C₂ to C₅)alkenyl, (C₁ to C₅ alkoxy) C₂ to C₅ alkenyl, C₂ to C₅ alkenylthio, C₂ to C₄ alkynyl, C₂ to C₅ alkynyloxy, C₂ to C₅ alkynylamino, di(C₂ to C₅ alkynyl)amino, C₂ to C₅ alkynylcarbonyl, C₂ to C₅ alkynyloxycarbonyl, C₂ to C₅ alkynylcarbonyloxy, halo(C₂ to C₅)alkynyl, amino(C₂ to C₅)alkynyl, hydroxy(C₂ to C₅)alkynyl, (C₁ to C₅ alkoxy) C₂ to C₅ alkynyl;
(ii) C₁ to C₅ alkoxy; and
(iii) aryl and arylalkyl.

2. A compound of formula I according to claim 1, wherein R1 represents a residue of the formula 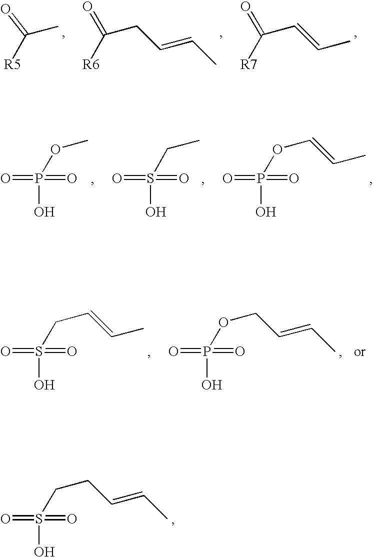
wherein R5, R6 and R7 are independently selected from H, OH, NR₂, NR₃, and R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl.

3. A compound according to claim 2 wherein g is 1 and R1 is

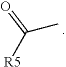

4. A compound according to claim 2 wherein g is 1 and R1 is

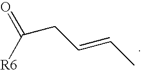

5. A compound according to claim 2 wherein R1 is

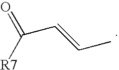

6. A compound of formula II 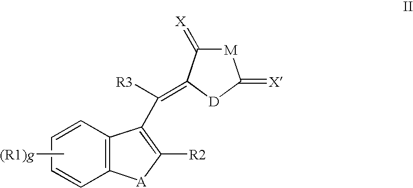
and pharmaceutically acceptable salts thereof, wherein
A, D and M are independently NR, 0 or S;
R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl;
g is 0, 1, 2, 3 or 4; and
X and X′ are independently O or S;
R1, R2 and R3 are independently selected from:
(i) hydrogen, C₁ to C₅ alkyl, OH, NH₂, C1 to C₅ alkylamino, di(C₁ to C₅ alkyl)amino, C₁ to C₅ alkylcarbonyl, C₁ to C₅ alkyloxycarbonyl, C₁ to C₅ alkylcarbonyloxy, carboxyl, C₁ to C₅ alkyl phosphonate, C₁ to C₅ alkenyl phosphonate, C₁ to C₅ alkyl phosphate, C₁ to C₅ alkenyl phosphate, C₁ to C₅ alkyl sulfonate, C₁ to C₅ alkenyl sulfonate, halo, halo(C₁ to C₅)alkyl, amino(C₁ to C₅)alkyl, hydroxyl(C₁ to C₅)alkyl, (C₁ to C₅)alkoxyl) C₁ to C₅ alkyl, NO₂, C₁ to C₅ alkylthio, SO₃H, PO₄, PO₃H, NH₄, C₂ to C₅ alkenyl, C₂ to C₅ alkenyloxy, C₂ to C₄ alkenylamino, di(C₂ to C₅ alkenylcarbonyl), C₂ to C5₄ alkenyloxycarbonyl, C₂ to C₄ alkylcarbonyloxy, halo(C₂ to C₅)alkynyl, amino(C₂ to C₅)alkenyl, hydroxy(C₂ to C₅)alkenyl, (C₁ to C₅ alkoxy) C₂ to C₅ alkenyl, C₂ to C₅ alkenylthio, C₂ to C₄ alkynyl, C₂ to C₅ alkynyloxy, C₂ to C₅ alkynylamino, di(C₂ to C₅ alkynyl)amino, C₂ to C₅ alkynylcarbonyl, C₂ to C₅ alkynyloxycarbonyl, C₂ to C₅ alkynylcarbonyloxy, halo(C₂ to C₅)alkynyl, amino(C₂ to C₅)alkynyl, hydroxy(C₂ to C₅)alkynyl, (C₁ to C₅ alkoxy) C₂ to C₅ alkynyl;
(ii) C₁ to C₅ alkoxy; and
(iii) aryl and arylalkyl.

7. A compound of formula II according to claim 6, wherein R1 represents a residue of the A compound of formula I according to claim 1, wherein R1 represents a residue of the formula 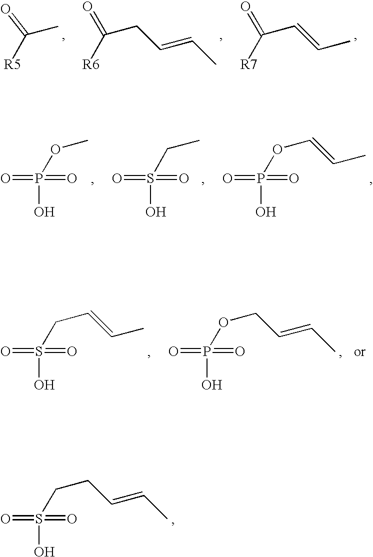
wherein R5, R6 and R7 are independently selected from H, OH, NR₂, NR₃, and R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl.

8. A compound according to claim 7 wherein g is 1 and R1 is

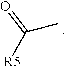

9. A compound according to claim 7 wherein g is 1 and R1 is

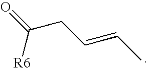

10. A compound according to claim 7 wherein g is 1 and R1 is

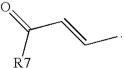

11. A compound according to claim 1 selected from the group consisting of:
1,2-dihydro-1-oxobenzofuro[2,3-c]pyridine-7-carboxylic acid;
1,2-dihydro-1-oxobenzofuro[2,3-c]pyridine-6-carboxylic acid;
(2E)-3-(1,2-dihydro-1-oxobenzofuro[2,3-c]pyridin-6-yl)acrylic acid; and
1,2-dihydro-1-oxobenzofuro[2,3-c]pyridine-8-carboxylic acid.

12. A compound according to claim 6 selected from the group consisting of:
(Z)-5-((1H-indol-3-yl)methylene)-2-thiooxazolidin-4-one;
(Z)-5-((1H-indol-3-yl)methylene)oxazolidine-2,4-dione;
(Z)-5-((1H-indol-3-yl)methylene)thiazolidine-2,4-dione;
(Z)-5-((1H-indol-3-yl)methylene)-2-thiothiazolidin-4-one, and
pharmaceutically acceptable salts thereof.

13. A method of treating a central nervous system (CNS) disorder associated with the striatal region of the brain, the method comprising:
administering an effective dose of a pharmaceutical formulation comprising a compound of formula I to a patient in need thereof exhibiting symptoms of a CNS disorder so as to attenuate said symptoms, wherein formula I is 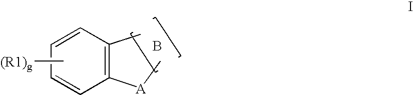
and pharmaceutically acceptable salts thereof, wherein
A is NR, O or S;
R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl;
g is 0, 1, 2, 3, or 4; and
B is a ring forming a fused ring system with the ring containing A and is selected from; 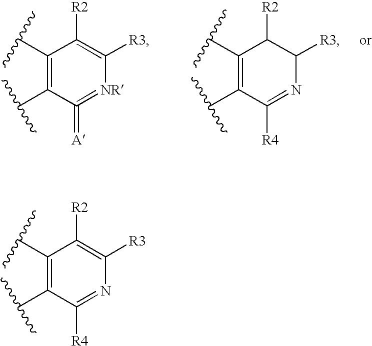
wherein A′ is as described above for A and NR′ is as described above for NR, R1, R2, R3 and R4 are independently selected from:
(i) hydrogen, C₁ to C₅ alkyl, OH, NH₂, C₁ to C₅ alkylamino, di(C₁ to C₅ alkyl)amino, C₁ to C₅ alkylcarbonyl, C₁ to C₅ alkyloxycarbonyl, C₁ to C₅ alkylcarbonyloxy, carboxyl, halo, halo(C₁ to C₅)alkyl, amino(C₁ to C₅)alkyl, hydroxyl(C₁ to C₅)alkyl, (C₁ to C₅)alkoxyl) C₁ to C₅ alkyl, NO₂, C₁ to C₅ alkylthio, SO₃H, PO₄, PO₃H, NH₄, C₂ to C₅ alkenyl, C₂ to C₅ alkenyloxy, C₂ to C₄ alkenylamino, di(C₂ to C₅ alkenylcarbonyl), C₂ to C5₄ alkenyloxycarbonyl, C₂ to C₄ alkylcarbonyloxy, halo(C₂ to C₅)alkynyl, amino(C₂ to C₅)alkenyl, hydroxy(C₂ to C₅)alkenyl, (C₁ to C₅ alkoxy) C₂ to C₅ alkenyl, C₂ to C₅ alkenylthio, C₂ to C₄ alkynyl, C₂ to C₅ alkynyloxy, C₂ to C₅ alkynylamino, di(C₂ to C₅ alkynyl)amino, C₂ to C₅ alkynylcarbonyl, C₂ to C₅ alkynyloxycarbonyl, C₂ to C₅ alkynylcarbonyloxy, halo(C₂ to C₅)alkynyl, amino(C₂ to C₅)alkynyl, hydroxy(C₂ to C₅)alkynyl, (C₁ to C₅ alkoxy) C₂ to C₅ alkynyl;
(ii) C₁ to C₅ alkoxy; and
(iii) aryl and arylalkyl.

14. A method for treating a CNS disorder according to claim 13, wherein the CNS disorder is psychosis, schizophrenia, or obsessive-compulsive disorder.

15. A method for modulating PDE10A expression in a subject, the method comprising:
administering a compound of formula I or formula II as claimed in either claim 1 or claim 6 in a pharmaceutical formulation;
measuring isolated PDE10A mRNA from a sample of blood from the patient using a quantitative replicative procedure such as QPCR; and
comparing the level of isolated mRNA from blood from the subject before and after administering the compound of formula II.

16. A method for modulating PDE10A according to claim 15, wherein the compound of formula I or formula II is selected from the group consisting of: 1,2-dihydro-1-oxobenzofuro[2,3-c]pyridine-7-carboxylic acid; 1,2-dihydro-1-oxobenzofuro[2,3-c]pyridine-6-carboxylic acid; (2E)-3-(1,2-dihydro-1-oxobenzofuro[2,3-c]pyridin-6-yl)acrylic acid; 1,2-dihydro-1-oxobenzofuro[2,3-c]pyridine-8-carboxylic acid; (Z)-5-((1H-indol-3-yl)methylene)-2-thiooxazolidin-4-one; (Z)-5-((1H-indol-3yl)methylene)oxazolidine-2,4-dione; (Z)-5-((1H-indol-3-yl)methylene)thiazolidine-2,4-dione; (Z)-5-((1H-indol-3-yl)methylene)-2-thiothiazolidin-4-one, and pharmaceutically acceptable salts thereof.

17. A method of inhibiting PDE10A according to claim 15, wherein modulating further comprises inhibiting.

18. A method for treating a central nervous system (CNS) disorder associated with the striatal region of the brain, the method comprising administering an effective dose of a pharmaceutical formulation comprising a compound of formula II to a patient in need thereof exhibiting symptoms of a CNS disorder so as to attenuate said symptoms, wherein formula II is 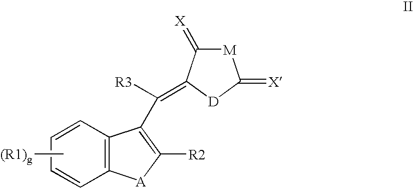
and pharmaceutically acceptable salts thereof, wherein
A, D and M are independently NR, O or S;
R is hydrogen, C₁ to C₅ alkyl, C₁ to C₅ acyl, C₁ to C₅ alkyloxycarbonyl, C₂ to C₅ alkenyl, C₂ to C₅ alkenylcarbonyl or C₂ to C₅ alkenyloxycarbonyl;
g is 0, 1, 2, 3 or 4; and
X and X′ are independently O or S;
R1, R2 and R3 are independently selected from:
(i) hydrogen, C₁ to C₅ alkyl, OH, NH₂, C₁ to C₅ alkylamino, di(C₁ to C₅ alkyl)amino, C₁ to C₅ alkylcarbonyl, C₁ to C₅ alkyloxycarbonyl, C₁ to C₅ alkylcarbonyloxy, carboxyl, halo, halo(C₁ to C₅)alkyl, amino(C₁ to C₅)alkyl, hydroxyl(C₁ to C₅)alkyl, (C₁ to C₅)alkoxyl) C₁ to C₅ alkyl, NO₂, C₁ to C₅ alkylthio, SO₃H, PO₄, PO₃H, NH₄, C₂ to C₅ alkenyl, C₂ to C₅ alkenyloxy, C₂ to C₄ alkenylamino, di(C₂ to C₅ alkenylcarbonyl), C₂ to C5₄ alkenyloxycarbonyl, C₂ to C₄ alkylcarbonyloxy, halo(C₂ to C₅)alkynyl, amino(C₂ to C₅)alkenyl, hydroxy(C₂ to C₅)alkenyl, (C₁ to C₅ alkoxy) C₂ to C₅ alkenyl, C₂ to C₅ alkenylthio, C₂ to C₄ alkynyl, C₂ to C₅ alkynyloxy, C₂ to C₅ alkynylamino, di(C₂ to C₅ alkynyl)amino, C₂ to C₅ alkynylcarbonyl, C₂ to C₅ alkynyloxycarbonyl, C₂ to C₅ alkynylcarbonyloxy, halo(C₂ to C₅)alkynyl, amino(C₂ to C₅)alkynyl, hydroxy(C₂ to C₅)alkynyl, (C₁ to C₅ alkoxy) C₂ to C₅ alkynyl;
(ii) C₁ to C₅ alkoxy; and
(iii) aryl and arylalkyl.

19. A method for treating a CNS disorder according to claim 18, wherein the CNS disorder is psychosis, schizophrenia, or obsessive-compulsive disorder.
